(19) United States
(12) Patent Application Publication
(10) Pub. No.: US 2026/0021299 A1
(43) Pub. Date: Jan. 22, 2026

(54) NEEDLE FOR IMPLANTATION OF LEAD FOR OBSTRUCTIVE SLEEP APNEA

(71) Applicant: MEDTRONIC, INC., MINNEAPOLIS, MN (US)

(72) Inventors: Phillip C. Falkner, Minneapolis, MN (US); Avram Scheiner, Vadnais Heights, MN (US); Adam J. Rivard, Blaine, MN (US); Sean P. Skubitz, Forest Lake, MN (US)

(21) Appl. No.: 18/998,776

(22) PCT Filed: Jun. 21, 2023
(86) PCT No.: PCT/US2023/025875

§ 371 Date: Jan. 27, 2025

Related U.S. Application Data
(60) Provisional application No. 63/369,309, filed on Jul. 25, 2022.

(51) Int. Cl. A61N 1/36; A61B 17/00; A61B 17/34; A61B 90/00; A61N 1/05; A61N 1/372

(52) U.S. Cl. CPC A61N 1/3601; A61B 17/3468; A61B 90/08; A61N 1/0502; A61N 1/0548; A61N 1/0558; A61N 1/3606; A61N 1/372; A61B 2017/00929; A61B 2090/0811

(57) ABSTRACT

This disclosure describes a system including a needle configured to percutaneously insert into skin and form a path for inserting a lead, the needle including a pointed distal end for percutaneously inserting the needle for placement near a hypoglossal nerve of a patient, and an elongated body comprising an inner lumen, wherein the elongated body defines one or more openings connecting an outer surface of the elongated body to the inner lumen. The lead may be configured to be disposed within the inner lumen of the elongated body and may include a shaft and one or more electrodes disposed on the shaft, configured to be placed near the hypoglossal nerve, and configured to stimulate the hypoglossal nerve for treating obstructive sleep apnea (OSA), wherein locations of the one or more electrodes on the shaft at least partially correspond to the one or more openings of the needle.

This application claims the benefit of U.S. Provisional Patent Application Ser. No. 63/369,309, filed Jul. 25, 2022, and entitled “NEEDLE FOR IMPLANTATION OF LEAD FOR OBSTRUCTIVE SLEEP APNEA,” the entire contents of which is incorporated herein by reference.

### TECHNICAL FIELD

This disclosure relates to medical device systems and, more particularly, to medical device systems for delivery of electrical stimulation therapy.

### BACKGROUND

Obstructive sleep apnea (OSA), which encompasses apnea and hypopnea, is a disorder in which breathing may be irregularly and repeatedly stopped and started during sleep, resulting in disrupted sleep and reduced blood oxygen levels. Muscles in a patient's throat intermittently relax thereby allowing soft tissues of the throat to obstruct the upper airway while sleeping and cause OSA. In patients with a smaller than normal airway, airflow into the upper airway can be obstructed by the tongue or soft pallet moving to the back of the throat and covering the airway. Loss of air flow also causes unusual inter-thoracic pressure as a person tries to breathe with a blocked airway. Lack of adequate levels of oxygen during sleep can contribute to abnormal heart rhythms, heart attack, heart failure, high blood pressure, stroke, memory problems, and increased accidents during the day due to inadequate sleep. Additionally, loss of sleep occurs when a person is awakened during an apneic episode.

### SUMMARY

The devices, systems, and techniques of this disclosure generally relate to an implantable medical device (IMD) system and methods for therapy for obstructive sleep apnea (OSA) but can be extended to address other patient symptoms and disorders. With OSA, a patient's tongue may relax during sleep and block the patient's airway. Some example techniques to address OSA include electrically stimulating one or both hypoglossal nerves and/or motor points in the tongue of the patient. In response to the electrical stimulation, the hypoglossal nerve(s) causes protrusor muscles (e.g., genioglossus and geniohyoid muscles) to contract and move the tongue forward, thereby opening the airway. In some examples, in response to stimulating at the motor points of the protrusor muscles (e.g., a location where an axon of the hypoglossal nerve terminates at a muscle fiber), the protrusor muscles may contract to move the tongue forward, thereby opening the airway.

To stimulate the hypoglossal nerve(s) and/or motor points, a medical device outputs electrical stimulation therapy via one or more electrodes on one or more implanted leads to cause the tongue to move forward. A medical professional can implant the one or more leads into the tongue of the patient using a needle. The one or more implanted leads each include one or more electrodes coupled to the medical device (e.g., an implantable or external medical device that delivers electrical stimulation via one or more electrodes on the lead).

To place a lead into the tongue of the patient, the medical professional may insert a needle into the tissue of the patient and near a target area (e.g., the hypoglossal nerve(s) and/or motor points). The medical professional may then insert a guidewire into the tissue of the patient through an inner lumen of the needle and remove the needle once the guidewire is in place. The medical professional may then advance an introducer sheath over the guidewire and the medical professional may then place the lead in an inner lumen of the introducer sheath, advance the lead through the introducer sheath, and remove the introducer sheath once the lead is in place. To perform the implantation process, the medical professional may need to use a plurality of tools (e.g., the needle, the guidewire, the introducer, and the like) and the implantation process may be time intensive.

This disclosure describes example needles and techniques of using needles to simplify the lead implantation process by retaining the example needles in the tissue of the patient and disposing the implantable leads within the example needles. Although the example techniques are described with respect to lead placement in the tongue for treating OSA, the example techniques should not be considered to be limited to lead placement in the tongue or limited to treating OSA.

In one example, the disclosure describes a system including a needle configured to percutaneously insert into skin and form a path for inserting a lead, the needle comprising: a pointed distal end for percutaneously inserting the needle for placement near a hypoglossal nerve of a patient; and an elongated body comprising an inner lumen, wherein the elongated body defines one or more openings connecting an outer surface of the elongated body to the inner lumen; and the lead configured to be disposed within the inner lumen of the elongated body, the lead comprising: a shaft; and one or more electrodes disposed on the shaft, configured to be placed near the hypoglossal nerve, and configured to stimulate the hypoglossal nerve for treating obstructive sleep apnea (OSA), wherein locations of the one or more electrodes on the shaft at least partially correspond to the one or more openings of the needle.

In one example, the disclosure describes a needle configured to percutaneously insert into skin and form a path for inserting a lead, the needle including a pointed distal end for percutaneously inserting the needle for placement near a hypoglossal nerve of a patient; an elongated body comprising an inner lumen, the elongated body defining one or more openings defined by the elongated body, wherein the one or more openings are configured to connect an outer surface of the elongated body to the inner lumen, wherein the elongated body is configured to place the lead disposed within the inner lumen near the hypoglossal nerve, the lead configured to stimulate the hypoglossal nerve for treating obstructive sleep apnea (OSA), and wherein the one or more openings are configured to at least partially correspond to locations of one or more electrodes positioned on a shaft of the lead.

In one example, the disclosure describes a method including percutaneously inserting a needle into skin of a patient, the needle comprising an elongated body comprising an inner lumen and defining one or more openings connecting an outer surface of the elongated body to the inner lumen: navigating the needle proximate to a hypoglossal nerve of the patient; inserting a lead into the inner lumen of the elongated body, the lead configured to stimulate the hypoglossal nerve for treating obstructive sleep apnea (OSA), the lead comprising: a shaft; and one or more electrodes disposed on the shaft, wherein locations of the one or more electrodes at least partially correspond to the one or more openings of the needle; and placing the one or more electrodes near the hypoglossal nerve.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

FIG. 1 is a conceptual diagram of an implantable medical device (IMD) system for delivering obstructive sleep apnea (OSA) therapy.

FIG. 2 is a conceptual diagram illustrating example locations of motor points where stimulation for OSA therapy may be delivered.

FIG. 3 is a block diagram illustrating example configurations of implantable medical devices (IMDs) which may be utilized in the system of FIG. 1.

FIG. 4 is a block diagram illustrating an example configuration of an external programmer.

FIG. 5 is a conceptual diagram illustrating an example needle assembly for the system of FIG. 1.

FIG. 6 is a conceptual diagram illustrating an example needle of the needle assembly of FIG. 5.

FIG. 7 is a conceptual diagram illustrating the implantable lead of the needle assembly of FIG. 5.

FIG. 8A is a conceptual diagram illustrating a cross-sectional view of an example electrode configuration of the needle assembly of FIG. 5 taken along line A-A.

FIG. 8B is a conceptual diagram illustrating a cross-sectional view of another example electrode configuration of the needle assembly of FIG. 5 taken along line A-A.

FIG. 8C is a conceptual diagram illustrating a cross-sectional view of another example electrode configuration of the needle assembly of FIG. 5 taken along line A-A.

FIG. 9 is a conceptual diagram illustrating a cross-sectional view of the needle assembly of FIG. 5 taken along line B-B.

FIG. 10 is a flowchart illustrating an example process of implanting an example needle assembly near the hypoglossal nerve(s).

### DETAILED DESCRIPTION

Medical devices, systems, and techniques for delivering electrical stimulation to the protrusor muscles of the tongue for the treatment of obstructive sleep apnea (OSA) are described in this disclosure. Electrical stimulation is delivered to cause the tongue of a patient to enter an advanced state, during sleep, to avoid or reduce upper airway obstruction. As used herein, the term, “advanced state” with regard to the tongue refers to a position that is moved forward and/or downward compared to a non-stimulated position or a relaxed position of the tongue. The advanced state is a state associated with contraction (e.g., via innervation from nerves in response to electrical stimulation) of protrusor muscles of the tongue (also sometimes referred to as “protruder” muscles of the tongue) including the genioglossus and geniobyoid muscles. An advanced state may be the opposite of a retracted and/or elevated position associated with the contraction of the retractor muscles (e.g., styloglossus and hyoglossus muscles) which retract and elevate the tongue. Electrical stimulation is delivered to cause the tongue to move (e.g., by depolarizing the nerve(s) that innervate the genioglossus and/or geniobyoid muscles) to and maintain an advanced state.

As discussed above, the advanced state may prevent collapse or blockage of, open, or widen the upper airway of a patient to at least partially maintain or increase airflow (e.g., promote unrestricted airflow or at least reduced restriction of airflow during breathing).

A surgeon implants one or more leads that each include one or electrodes into the tongue such that the electrodes are proximate to a hypoglossal nerve and/or motor points (e.g., one or more locations where axons of the hypoglossal nerve terminate at respective muscle fibers of the protrusor muscles). For example, there are two hypoglossal nerves in the tongue of the patient. In one example, one lead may be used to stimulate (e.g., by delivering electrical stimulation through one or more electrodes of the lead) one of the two hypoglossal nerves, one lead may be used to stimulate both hypoglossal nerves, or two leads may be used, where each lead stimulates a respective one of the hypoglossal nerves. Stimulation of either or both hypoglossal nerves of the tongue can cause contraction of the protrusor muscles to reduce the effect of or prevent OSA.

There are multiple sets of motor points for each of the protrusor muscles on the left side and the right side. Each motor point may innervate one or more muscle fibers of the protrusor muscle. In one example, one lead may be used to stimulate motor points for the protrusor muscles on one side of the tongue, one lead may be used to stimulate motor points for protrusor muscles on both sides of the tongue, or two leads may be used, where each lead stimulates a respective set of motor points for the protrusor muscles on each side. Stimulation of either or both sets of motor points of the tongue can cause contraction of the protrusor muscles to reduce the effect of, or prevent, OSA.

This disclosure describes examples of techniques related to implantation of the one or more leads in the tongue for treatment of OSA. Although the example techniques are described with respect to OSA, the example techniques should not be construed as limited to OSA. Rather, the example techniques described in this disclosure may be applicable to lead implantation for treatment of various conditions, including lead implantation for treatment of conditions where the lead is implanted in a location other than the tongue.

Open surgeries may be performed to implant the one or more leads in a tongue of a patient for treating OSA. However, such open surgeries require dissection of tissue to expose one or more hypoglossal nerves and/or motor points for placement of the one or more leads immediately adjacent to or around the hypoglossal nerves and/or motor points in the tongue of the patient, which is relatively invasive and time-consuming.

In other examples, medical professionals may implant the leads by using a needle to form a path through the tissue of the patient to the hypoglossal nerves of the patient. The one or more leads may then be navigated through the paths to areas adjacent to the hypoglossal nerves and deliver stimulation signals to the hypoglossal nerves. In such examples, the medical professional may first create an initial path using a needle and remove the needle from the patient once the initial path has been created. The medical professional may then advance an introducer attached to a dilator, e.g., over a guidewire or other similar guiding device, to dilate the initial path to an appropriate diameter for the lead and to determine the appropriate orientation for the electrodes of the lead. The medical professional may then remove to the dilator and introducer from the patient and advance an implantable lead sheath into the dilated path. The implantable lead sheath may include an electrically insulative material and may be configured to electrically insulate some portions of the lead while allowing other portions of the lead to deliver stimulation signals to the hypoglossal nerve, e.g., through the one or more electrodes. Finally, the medical professional may insert the lead into an inner lumen of the implantable lead sheath and advance the lead through the inner lumen of the implantable lead sheath to the hypoglossal nerves of the patient to complete the implantation process.

Unlike the examples that require dissecting tissue or examples that require a needle, introducer, guidewire, and other such components, the example techniques described in this disclosure utilize a needle configured to percutaneously insert into skin and form a path for inserting a lead, such as without requiring the use of the guidewire or introducer. For instance, the example techniques described in this disclosure may enable a surgeon to implant one or more leads adjacent to or around one or more hypoglossal nerves and/or motor points in the tongue of a patient without dissecting tissue to expose the hypoglossal nerves and/or motor points, which minimize access incision, shorten recovery time for the patient, and reduce risk for misplacement of the leads. In addition, the example techniques described in this disclosure may enable a surgeon to implant the one or more leads adjacent to or around one or more hypoglossal nerves and/or motor points in the tongue of the patient with fewer tools and complete the lead implantation process in a shorter duration. For example, by using a needle with one or more openings at least partially corresponding to one or more electrodes positioned on the lead, as described in greater detail below, a surgeon may determine the appropriate orientation and stimulation signal of the one or more electrodes of the lead without requiring the use of another device, e.g., an introduce needle.

In some examples, the needle described in this disclosure may form and dilate the path leading to the hypoglossal nerves, thereby eliminating the requirement for a dilator during the lead implantation process. In some examples, the needle described in this disclosure may perform the functions of the implantable lead sheath by using the one or more openings of the needle.

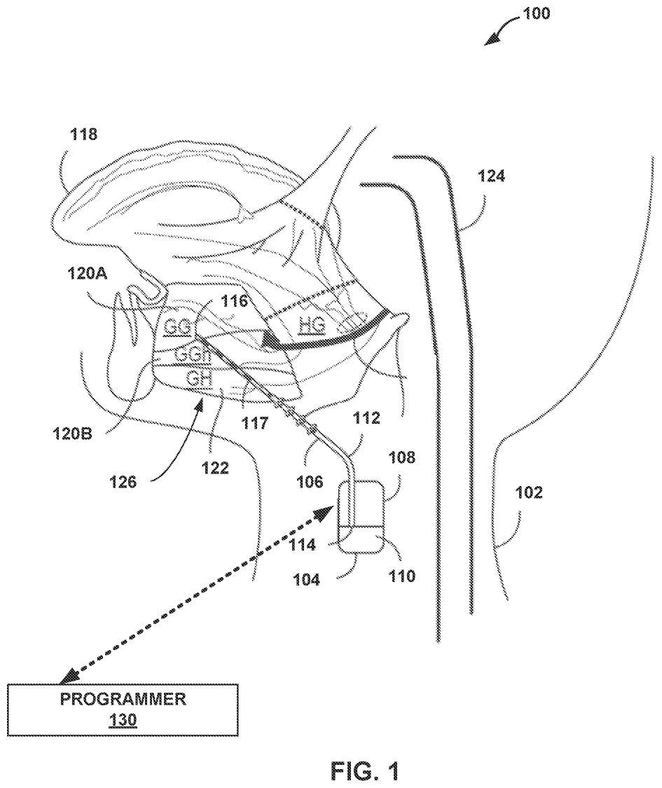

FIG. 1 is a conceptual diagram of a medical system for delivering OSA therapy. In system 100, implantable medical device (IMD) 104 and lead 106 are implanted in patient 102. IMD 104 includes housing 108 enclosing circuitry of IMD 104. In some examples, IMD 104 includes connector assembly 110, which is hermetically sealed to housing 108 and includes one or more connector bores for receiving a proximal end of at least one medical electrical lead 106, also called lead 106, used for delivering OSA therapy. Although one lead 106 is illustrated in FIG. 1, there may be one or more leads 106 to which IMD 104 is coupled.

Lead 106 may include a flexible, elongated lead body 112, also called elongated member 112, that extends from lead proximal end 114 to lead distal end 116. As illustrated, lead 106 includes one or more electrodes 117 that are carried along a lead distal portion adjacent lead distal end 116 and are configured for insertion within the protrusor muscles 120A, 120B, and 122 of tongue 118. As one example, the genioglossus muscle includes oblique compartment 120A and horizontal compartment 120B. In this disclosure, the genioglossus muscle is referred to as protrusor muscle 120. Protrusor muscle 122 is an example of the geniohyoid muscle.

As illustrated, distal end 116 of lead 106 includes one or more electrodes 117. Proximal end 114 of lead 106 includes one or more electrical contacts to connect to connector assembly 110. Lead 106 also includes conductors such as coils or wires that connect respective electrodes 117 to respective electrical contacts at proximal end 114 of lead 20.

During implantation of system 100, a medical professional may insert a needle within the protrusor muscles 120A, 120B, and 122 of tongue 118. The medical professional may then place lead 106 within protrusor muscles 120 and/or 122 through an inner lumen of the needle. The medical professional may remove the needle from tongue 118 once lead 106 is positioned within protrusor muscles 120 and/or 122. In some examples, the medical professional may advance lead 106 within the needle and deliver test stimulation signals to protrusor muscles 120 and/or 122 via lead 106 (e.g., through one or more openings in the needle) and/or the needle. System 100 may sense electrical signals (e.g., evoked electrical signals) from the tissue of patient 102 in response to the test stimulation signals. The medical professional may determine, based on the sensed electrical signals, if lead 106 is placed in a proper location within protrusor muscles 120 and/or 122 to deliver stimulation to hypoglossal nerve(s) and/or motor points of patient 102. The medical professional may iteratively re-position the needle and lead 106 and transmit test stimulation signals until the medical professional determines that lead 106 is properly placed within protrusor muscles 120 and/or 122. The medical professional may then retract the needle and leave lead 106 in place. Once the needle is retracted and removed from the body of patient 102, the medical professional may implant the remainder of system 100 (e.g., IMD 104) and complete the implantation process.

The needle may include a plurality of openings connecting an inner lumen within the needle to an outer surface of the needle. Lead 106 may be disposed within the inner lumen of the needle. Electrodes 117 of lead 106 may correspond to at least some of the plurality of openings of the needle. Although one needle is illustrated in FIG. 1, the medical professional may use a plurality of needles (e.g., two or more needles) during the implantation process. The number of needles may correspond to the number of leads 106 to which IMD 104 is coupled.

While protrusor muscles 120 and 122 are described, the example techniques described in this disclosure are not limited to stimulating protrusor muscles 120 and 122. Also, FIG. 1 illustrates one set of protrusor muscles 120 and 122 (e.g., on a first side of tongue 118). The other side of tongue 118 also includes protrusor muscles. For instance, a left side of tongue 118 includes a first set of protrusor muscles 120 and 122, and a right side of tongue 118 includes a second set of protrusor muscles.

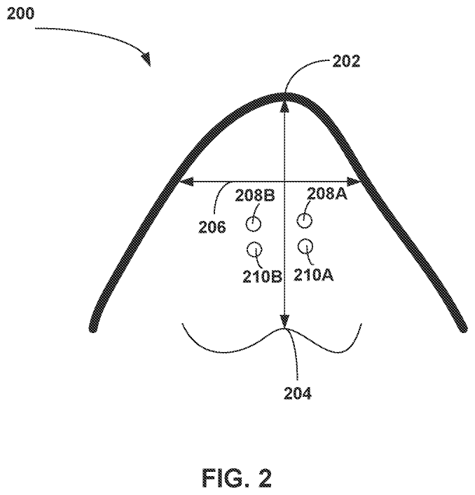

In some examples, a medical professional may implant one or more leads 106 and one or more needles such that one or more electrodes 117 are implanted within soft tissue, such as musculature, proximate to medial branches of one or both hypoglossal nerves. In some examples, one or more electrodes 117 may be approximately 5 mm (e.g., 2 mm to 8 mm) from a major trunk of the hypoglossal nerve. In some examples, one or more electrodes 117 may be placed in an area of protrusor muscles 120 and 122 that include motor points, where each nerve axon terminates in the muscle (also called the neuro-muscular junction). The motor points are not at one location but spread out in the protrusor muscles. Leads 106 may be implanted such that one or more electrodes 117 may be generally in the area of the motor points (e.g., such that the motor points are within 1 to 10 mm from one or more electrodes 117). Examples of motor points for protrusor muscles 120 and 46 are illustrated in more detail with respect to FIG. 2.

Tongue 118 includes a distal end (e.g., tip of tongue 118), and electrodes 117 may be implanted proximate to root 126 of tongue 118. The surgeon may implant one or more leads 106 such that one or more electrodes are implanted proximate to root 126 of tongue 118, as illustrated in FIG. 1. For example, the location for stimulation for the genioglossus muscle 120 may be approximately 30 mm (e.g., 25 mm to 35 mm) from the symphysis of the jaw (e.g., where the genioglossus and hypoglossal muscles insert). The location for stimulation for the geniohyoid muscle 122 may be approximately 40 mm (e.g., 35 mm to 45 mm) from the symphysis. For both the genioglossus muscle 120 and the geniohyoid muscle 122, the location for stimulation may be approximately 11 mm (e.g., 7 mm to 15 mm) lateral to the midline on both the right and left sides of tongue 118 for stimulating respective hypoglossal nerves. In some examples, rather than stimulating hypoglossal nerves, the examples described in this disclosure may be configured for stimulating the motor points, as described in more detail with respect to FIG. 2. Stimulating the motor points may result in indirect activation of the hypoglossal nerve, but may generally be stimulating at a different location than direct stimulation to the hypoglossal nerve. As a result, in some examples, simulation of one or more motor points may result in more precise activation of muscle fibers than may be possible with stimulation of the hypoglossal nerve itself.

One or more electrodes 117 of lead 106 may be ring electrodes, segmented electrodes, partial ring electrodes, or any suitable electrode configuration. Ring electrodes extend 360 degrees around the circumference of lead body 112 of lead 106. Segmented and partial ring electrodes each extend along an are less than 360 degrees (e.g., 90-120 degrees) around the outer circumference of lead body 112 of lead 106. In this manner, multiple segmented electrodes may be disposed around the perimeter of lead 106 at the same axial position of the lead. In some examples, segmented electrodes may be useful for targeting different fibers of the same or different nerves at respective circumferential positions with respect to the lead to generate different physiological effects (e.g., therapeutic effects), permitting stimulation to be oriented directionally. In some examples, lead 106 may be, at least in part, paddle-shaped (e.g., a “paddle” lead), and may include an array of electrodes arranged as contacts or pads on a common surface, which may or may not be substantially flat and planar.

As described above, in some examples, electrodes 117 of lead 106 are disposed within the musculature of tongue 118. Accordingly, one or more electrodes 117 of lead 106 may be “intramuscular electrodes.” Intramuscular electrodes may be different than other electrodes that are placed on or along a nerve trunk or branch, such as a cuff electrode, used to directly stimulate the nerve trunk or branch. The example techniques described in this disclosure are not limited to intramuscular electrodes and may be extendable to electrodes placed closer to a nerve trunk or branch of the hypoglossal nerve(s). Also, in some examples, one or more electrodes 117 of lead 106 may be implanted in connective tissue or other soft tissue proximate to the hypoglossal nerve.

In some examples, the needle may be configured for advancement through the soft tissue, which may include the protrusor muscle tissue, to anchor lead 106 and electrodes 117 of lead 106 in proximity to the hypoglossal nerve(s) that innervate protrusor muscles 120 and/or 122 and/or motor points that connect axons of hypoglossal nerve(s) to respective muscle fibers of protrusor muscles 120 and/or 122. In some examples, the needle is used for vascular implantation. In such examples, the needle may include a hemostasis valve positioned within an attachment member disposed on a proximal portion of the needle assembly. The hemostasis valve may prevent transfer of blood or other bodily fluids into the needle assembly.

As described above, electrical stimulation therapy generated by IMD 104 and delivered via one or more electrodes 117 may activate protrusor muscles 120 and 122 to move tongue 118 forward, for instance, to promote a reduction in obstruction or narrowing of the upper airway 124 during sleep. As used herein, the term “activated” with regard to the electrical stimulation of protrusor muscles 120 and 122 refers to electrical stimulation that causes depolarization or an action potential of the cells of the nerve (e.g., hypoglossal nerve(s)) or stimulation at the neuro-muscular junction between the nerve and the protrusor muscles (e.g., at the motor points) innervating protrusor muscles 120 and 122 and motor points and subsequent depolarization and mechanical contraction of the protrusor muscle cells of protrusor muscles 120 and 122. In some examples, protrusor muscles 120 and 122 may be activated directly by the electrical stimulation therapy.

Protrusor muscles 120 and/or 122, on a first side of tongue 118 (e.g., the left or right side of tongue 118), may be activated by a medial branch of a first hypoglossal nerve, and the protrusor muscles, on a second side of tongue 118 (e.g., the other of the left or right side of tongue 118), may be activated by a medial branch of a second hypoglossal nerve. The medial branch of a hypoglossal nerve may also be referred to as the XIIth cranial nerve. The hyoglossus and styloglossus muscles (not shown in FIG. 1), which cause retraction and elevation of tongue 118, are activated by a lateral branch of the hypoglossal nerve.

One or more electrodes 117 may be used to deliver bilateral or unilateral stimulation to protrusor muscles 120 and 122 via the medial branch of the hypoglossal nerve or branches of the hypoglossal nerve (e.g., such as at the motor point where a terminal branch of the hypoglossal nerve interfaces with respective muscle fibers of protrusor muscles 120 and/or 122). For example, one or more electrodes 117 may be coupled to output circuitry of IMD 104 to enable delivery of electrical stimulation pulses in a manner that selectively activates the right and left protrusor muscles (e.g., in a periodic, cyclical, or alternating pattern) to avoid muscle fatigue while maintaining upper airway patency. Additionally, or alternatively, IMD 104 may deliver electrical stimulation to selectively activate protrusor muscles 120 and/or 122 or portions of protrusor muscles 120 and/or 122 during unilateral stimulation of the left or right protrusor muscles.

In some examples, one lead 106 may be implanted such that one or more of electrodes 117 deliver electrical stimulation to stimulate the left hypoglossal nerve or motor points of protrusor muscles on the left side of tongue, and therefore cause the left protrusor muscles to activate. In such examples, the electrical stimulation from one or more electrodes 117 may not be of sufficient amplitude to stimulate the right hypoglossal nerve or motor points of protrusor muscles on the right side of tongue and cause the right protrusor muscles to activate. In some examples, one lead 106 may be implanted such that one or more of electrodes 117 deliver electrical stimulation to stimulate the right hypoglossal nerve or motor points of protrusor muscles on the right side of tongue, and therefore cause the right protrusor muscles to activate. In such examples, the electrical stimulation from one or more electrodes 117 may not be of sufficient amplitude to stimulate the left hypoglossal nerve or motor points of protrusor muscles on the left side of tongue and cause the left protrusor muscles to activate. Accordingly, in some examples, two leads like lead 106 may be implanted to stimulate each of the left and right hypoglossal nerves and/or motor points of respective protrusor muscles on the left and right side of tongue 118.

In some examples, one lead 106 may be implanted substantially in the middle (e.g., center) of tongue 118. In such examples, one or more electrodes 117 may deliver electrical stimulation to both hypoglossal nerves or motor points of both muscles on the both sides of tongue 118, causing both hypoglossal nerves or motor points to activate respective left and right protrusor muscles. It may be possible to utilize current steering and field shaping techniques such that one or more electrodes 117 deliver first electrical stimulation that stimulates the left hypoglossal nerve or motor points of protrusor muscles on the left side of tongue 118 with little to no stimulation of the right hypoglossal nerve or motor points of protrusor muscles on the right side of tongue 118, and then one or more electrodes 117 deliver second electrical stimulation that stimulates the right hypoglossal nerve or motor points of protrusor muscles on the right side of tongue with little to no stimulation of the left hypoglossal nerve or motor points of protrusor muscles on the left side of tongue. In examples where two leads like lead 106 are utilized, each lead may alternate delivery of stimulation to respective hypoglossal nerves or motor points. In this way, IMD 104 may stimulate one hypoglossal nerve or one set of motor points and then the other hypoglossal nerve or another set of motor points, which can reduce muscle fatigue.

For instance, continuous stimulation may cause protrusor muscles to be continuously in an advanced state. This continuous contraction may cause protrusor muscles 120 and/or 122 to fatigue. In such cases, due to fatigue, the stimulation may not cause protrusor muscles 120 and/or 122 to maintain an advanced state (or higher intensity of the electrical stimulation may be needed to cause protrusor muscles 120 and/or 122 to remain in the advanced state). By stimulating one set of protrusor muscles (e.g., left or right), a second set (e.g., other of left or right) of protrusor muscles can be at rest. Stimulation may then alternate to stimulate the protrusor muscles that were at rest and thereby maintain protrusion of tongue 118 while permitting the protrusor muscles 120 and/or 122 that were previously activated to rest. Hence, by cycling between alternate stimulation of the left and right protrusor muscles, tongue 118 can remain in the advanced state, while one of the first or second set of protrusor muscles is at rest.

In some examples, one lead 106 may be implanted laterally or diagonally across tongue 40 such that some of electrodes 117 on lead 106 can be used to stimulate the left hypoglossal nerve and/or motor points of the protrusor muscles on the left side of tongue 118 and some of electrodes 117 on the same lead 106 can be used to stimulate the right hypoglossal nerve and/or motor points of the protrusor muscles on the right side of tongue 118. In such examples, IMD 104 may selectively deliver electrical stimulation to a first hypoglossal nerve and/or first motor points of the protrusor muscles on a first side of tongue 118 via a first set of one or more electrodes 117, and then deliver electrical stimulation to a second hypoglossal nerve and/or/or second set of motor points of the protrusor muscles on a second side of tongue 118 via a second set of one or more electrodes 117. This may be another way in which to reduce muscle fatigue.

Lead proximal end 114 includes a connector (not shown in FIG. 1) that may be coupled to connector assembly 110 of IMD 104 to provide electrical connection between circuitry enclosed by the housing 108 of IMD 104. Lead body 112 encloses electrical conductors extending from each of one or more electrodes 117 to the proximal connector at proximal end 114 to provide electrical connection between output circuitry of IMD 104 and the electrodes 117.

There may be various ways in which lead 106 is implanted in patient 102. A surgeon may insert the needle through the lower part of the jaw and in tongue 118 starting from the back of tongue 118. The surgeon may insert the needle until a distal tip of the needle reaches a point at or adjacent to root 126 of tongue 118, angling the needle to extend proximate to the hypoglossal nerve (e.g., left or right hypoglossal nerve). In some examples, the needle may include one or more electrically conductive areas (e.g., one or more electrodes) at the distal end, and the surgeon may cause the one or more electrically conductive areas of the needle to output electrical stimulation (e.g., in the form of controlled current pulses or controlled voltage pulses), which in turn causes a physiological response such as activation of protrusor muscles 120 and/or 122 and protrusion of tongue 118. In some examples the one or more electrodes may be disposed on an outer surface of the needle. The surgeon may adjust the location of the needle based on the physiological response to determine a location in tongue 118 that provides effective treatment. Using the needle with stimulating electrodes is not necessary in every example. In some examples, once the needle is implanted within tongue 118, the surgeon may advance lead 106 through the inner lumen of the needle. In some examples, the surgeon may position lead 106 in a first position where one or more electrodes 117 of lead 106 are occluded by the needle. In some examples, the surgeon may position lead 106 in a second position within the inner lumen of the needle where one or more electrodes 117 of lead 106 at least partially correspond to the one or more openings within the needle.

IMD 104 may output stimulation signals through electrodes 117 and the one or more openings within the needle to stimulate the hypoglossal nerve and/or one or more motor points of the protrusor muscle within tongue 118. If further refinement is needed to determine the lead placement for lead 106, the surgeon may adjust the location of lead 106 within the needle in response to one or more electrical signals detected by electrodes 117 and/or electrodes on the needle. The needle may be appropriately sized to receive lead 106. During implantation and testing of lead 106, IMD 104 may not yet be implanted within body of patient 102. After completing implantation and testing of lead 106, the medical professional may implant IMD 104 within patient 102 (e.g., in the neck of patient 102, in the torso of patient 102, or the like) to complete the implantation of system 100. In some examples, lead 106 may be connected to another computing device and/or system (e.g., an external programming device) and the another computing device and/or system may output the stimulation signals for purposes of delivering the lead placement for lead 106.

In one or more examples, because the needle is appropriately sized to receive lead 106, implantation of lead 106 may not require the use of an introducer sheath or a guidewire. In this way, the example techniques may reduce components and surgical complexity for implanting lead 106.

As an example, some other techniques of implanting lead 106 include using a needle (not sized to receive lead 106) to percutaneously insert into the skin. A surgeon places a guidewire through the lumen of needle, then removes the needle. The guidewire remains in place in the tissue in original location as the needle inside the patient. The surgeon places an introducer sheath, possibly with a dilator, over the guidewire. The surgeon removes the guidewire, and places lead 106 include the introducer sheath. The surgeon then removes the introducer sheath leaving lead 106 in place.

The needle described in this disclosure may reduce the implantation complexity. For instance, the surgeon may percutaneously insert the needle to form a path for lead 106. A separate probe may not be needed to locate the implant site because the needle can be used for unipolar stimulation. Furthermore, in some examples, the needle may include electrodes on the needle to deliver electrical stimulation, and/or may include openings to allow electrodes 117 of lead 106 to deliver electrical stimulation after lead 106 is placed in the lumen of the needle. Accordingly, in some examples, a guidewire and introducer sheath may not be needed for implantation. Because the needle is appropriately sized to receive lead 106, the surgeon may directly insert lead 106 into the lumen of the needle. In this way, the needle may facilitate accurate placement of lead 106, such as by ensuring that when electrical stimulation is delivered at the location of the needle, tongue 108 moves to the advanced state.

For instance, the surgeon may put lead 106 through the needle such that one or more electrodes 117 are proximate to the hypoglossal nerve (e.g., such that distal end 116 is near tip of tongue as one non-limiting example). Electrodes 117 may be proximate to the hypoglossal nerve and/or motor points of the protrusor muscles 120 and/or 122 due to the needle creating an opening near the hypoglossal nerve and/or motor points of the protrusor muscle. In some examples, the surgeon may tunnel proximal end 114 of lead 106 back to a connection with IMD 104.

In this manner, the surgeon may implant one lead 106. In examples where two or more leads are implanted, the surgeon may perform steps similar to those described above.

The above describes some example techniques for lead placement, and the examples described in this disclosure should not be considered limited to such examples of lead placement. Moreover, in some examples, the surgeon may use imaging techniques, such as fluoroscopy, during implantation to verify proper placement of lead 106, the introducer needle, and/or the introducer sheath.

FIG. 1 illustrates the location of IMD 104 as being within or proximate to the neck of patient 102. However, IMD 104 may be implanted in various other locations. As one example, the surgeon may implant IMD 104 in the left or right pectoral region. For instance, the surgeon may plan on implanting IMD 104 in the left pectoral region unless another medical device is already implanted in the left pectoral region. If another medical device is already implanted in the left pectoral region, the surgeon may then implant IMD 104 in the right pectoral region. There may include other locations where the surgeon may implant IMD 104, such as the back of patient 102. The example techniques are not limited to any particular implant location of IMD 104.

In accordance with one or more examples described in this disclosure, system 100 is an implant system for utilizing lead 106 in tongue 118 for treatment of OSA. In some examples, system 100 may be configured such that substantial dissection is not required to expose one or more hypoglossal nerves and/or one or more motor points of the protrusor muscle within tongue 118 for placement of the lead. In some examples, system 100 may be configured such that a surgeon may implant the needle and lead 106 within patient 102 using a relatively smaller number of devices (e.g., without the use of an introducer sheath, guide members (e.g., a guidewire), a dilator, and the like). This disclosure describes examples of system 100 configured for placement of lead 106 in a way that minimizes access incisions for placement of lead 106.

In some situations, it may be desirable to include multiple electrodes on lead 106 to achieve desired physiological effects (e.g., therapeutic effects). For example, to achieve the desired effect, multiple electrodes may be used to target different fibers of the same nerve (e.g., target one or more motor points of the protrusor muscle within tongue 118). In such cases, determining the locations of the different fibers or motor points one at a time is time-consuming and may cause nerve injury. In some examples, system 100 may enable a surgeon to identify the locations of different fibers or motor points of the protrusor muscles in such a manner to shorten the surgical time and reduce the risk of nerve injury.

As described above, system 100 is an implant system for implanting lead 106 adjacent to or around one or more hypoglossal nerves and/or motor points without open surgery, so that lead 106 may be implanted to stimulate the nerves with minimal impact to patient 102. There may be certain unique challenges associated with implanting lead 106 adjacent to or around the hypoglossal nerves and/or the one or more motor points of the protrusor muscle (e.g., protrusor muscles 120 and/or 122) in tongue 118 without open surgery. As one example, without performing an open surgery to expose the hypoglossal nerves and/or motor points, there are difficulties with localizing and accessing the hypoglossal nerves and/or motor points.

To identify the location of a hypoglossal nerve and/or a motor point without performing an open surgery, system 100 may include the needle for creating an opening in tongue 118 of the patient for implantation of lead 106 and a medical device for delivering stimulation signals through lead 106 and the needle to tongue 118 of patient 102 to stimulate the hypoglossal nerve and/or the motor point. The same medical device or possibly another medical device may further receive electrical signals from lead 106, where the electrical signals (e.g., EMG signals) are generated from a muscle movement in response to the stimulation signals. As illustrated in FIG. 1, the medical device may be an implantable medical device (e.g., IMD 104) implanted near the neck of patient 102. Hence, IMD 104 may be utilized for chronic (i.e., long-term) treatment of OSA. However, in some examples, during the implantation of lead 106 or determining location for implanting lead 106, a trial stimulator (e.g., external medical device) may be used to deliver stimulation to the needle or through lead 106 when lead 106 is within the needle. Accordingly, the medical device may be an external medical device coupled to lead 106 and/or the needle for delivering and/or detecting stimulation signals. It should be noted that IMD 104 may also be used as a trial stimulator, and the techniques are not limited to an external medical device.

System 100 includes an needle that has an elongated body (e.g., the needle body). The elongated body may define an inner lumen within the needle and one or more openings extending from an outer surface of the elongated body to the inner lumen. The elongated body may be a malleable elongated body so that a surgeon can bend the desired shape for properly introducing lead 106. In some examples, the elongated body may be steerable so the surgeon can align lead 106 in a proper configuration intraoperatively. Having steerability in the elongated body may make it easier for surgeons to deploy lead 106 alongside and in proximity to a hypoglossal nerve and/or a motor point in tongue 118 of patient 102.

In some examples, the needle may include one or more electrodes positioned on the outer surface of the elongated body. The one or more electrodes positioned on the outer surface of the elongated body are different than one or more electrodes 117, where electrodes 117 are on lead 106. The one or more electrodes may be configured to deliver electrical stimulation signals to tongue 118 of patient 102 and/or detect electrical signals from tongue 118. Electrodes configured to deliver electrical stimulation may also be referred herein as “stimulation electrodes” and electrodes configured to detect electrical signals may also be referred herein as “sensing electrodes.”

System 100, along with the needle and lead 106, also includes a medical device for delivering stimulation signals via the needle through one or more stimulation electrodes on the needle to tongue 118 of patient 102 to stimulate a hypoglossal nerve and/or a motor point in tongue 118 of patient 102 as part of the implantation procedure. The medical device may also receive one or more electrical signals detected by the needle through one or more sensing electrodes and output information indicative of the one or more electrical signals. For example, the medical device may receive an EMG signal that measures an electrical current generated from a muscle contraction in response to the stimulation signal.

For instance, a surgeon may insert the needle in tongue 118 of patient 102 such that one or more electrodes on the needle are pushed through tissue near a chin of patient 102 and through tongue 118 proximate to the hypoglossal nerve and/or the motor point of a protrusor muscle (e.g., protrusor muscles 120 and/or 122) within tongue 118. After inserting the needle, the surgeon may control the medical device to deliver a stimulation signal via the needle through the one or more stimulation signals to tongue 118 of patient 102 to stimulate the hypoglossal nerve and/or the motor point in tongue 118 of patient 102. The surgeon may also control the medical device (same or different medical device) to receive electrical signals detected by the needle through one or more sensing electrodes and output information indicative of the one or more electrical signals on a display device. The surgeon may then determine a target treatment site based on the output information indicative of the one or more electrical signals.

In some examples, the one or more stimulation electrodes and/or the one or more sensing electrodes of the needle may be disposed between one or more openings on the elongated body of the needle. The one or more stimulation electrodes and/or the one or more sensing electrodes may be placed on a portion of the elongated body between two adjacent openings of the one or more openings. In other examples, the one or more stimulation electrodes and/or the one or more sensing electrodes may be placed on the elongated body of the needle at one or more different axial positions than the one or more openings. For example, the electrodes may be positioned proximally to the one or more openings, distally to the one or more openings, or proximal to a first set of one or more openings and distal to a second set of one or more openings on the elongated body.

The inner lumen of the needle has a sufficiently large diameter such that lead 106 may be disposed within the inner lumen of the needle and may be moved axially and/or rotated within the inner lumen of the needle.

After insertion of lead 106 into the needle, the surgeon may control a medical device (e.g., IMD 104) to output stimulation signals through electrodes 117 of lead 106 and one or more openings of the needle to stimulate the hypoglossal nerve and/or the motor point to determine the optimal placement of lead 106. The medical professional may remove the needle after implantation of lead 106 and lead 106 may then deliver chronic stimulation to patient 102.

FIG. 2 is a conceptual diagram illustrating example locations of motor points where stimulation for OSA therapy may be delivered. FIG. 2 illustrates jaw 200 of patient 102, where patient 102 is in a supine position and jaw 200 of patient 102 is viewed from an inferior location of patient 102. For instance, FIG. 2 illustrates symphysis 202 and hyoid bone 204. In the example illustrated in FIG. 2, the line interconnecting symphysis 202 and hyoid bone 204 may be considered as a y-axis along the midline of tongue 118. FIG. 2 also illustrates intergonial distance 206 between the two gonia of patient 102, where the gonia is a point on each side of the lower jaw 200 at the mandibular angle. Intergonial distance 206 may be along the x-axis of tongue 118.

FIG. 2 illustrates motor points 208A and 208B and motor points 210A and 210B. Motor points 208A may be motor points for the right genioglossus muscle, and motor points 208B may be motor points for the left genioglossus muscle. Motor points 210A may be motor points for the right geniohyoid muscle, and motor points 210B may be motor points for the left geniohyoid muscle. Motor points 208A and 208B and motor points 210A and 210B may genericize the motor points for each muscle for purposes of illustration. There may be additional motor points and/or motor points at different locations for each muscle.

In one or more examples, the needle, lead 106, and/or one or more electrodes 117 may be implanted proximate to motor points 208A, 208B, 210A, or 210B for stimulating at motor points 208A, 208B, 210A, and/or 210B. For instance, in examples where two leads are implanted, a first lead and its electrodes may be implanted proximate to motor points 208A and/or 210A and a second lead and its electrodes may be implanted proximate to motor points 208B and/or 210B. In one or more examples, electrodes 117 may be approximately 1 mm to 10 mm from respective motor points 208A, 208B, 210A, or 210B.

A hypoglossal nerve (e.g., on the left or right side of tongue 118) initially is a trunk of nerves fibers called axons. The axons of the hypoglossal nerve branch out. For example, the trunk of hypoglossal nerve includes multiple sets of axons including a first set of axons, and the first set of axons branch out from the trunk of the hypoglossal nerve. The first set of axons include multiple groups of axons including a first group of axons, and the first group of axons branch out from the first set of axons, and so forth. The locations where the branched-out axons interface with respective muscle fibers of protrusor muscles 120 and/or 122 (e.g., genioglossus and/or geniohyoid muscle) are referred to as motor points.

For instance, a branch of the hypoglossal nerve that interfaces (e.g., connects at the neuro-muscular junction) with the muscle fiber is referred to as a terminal branch, and the end of the terminal branch is a motor point. The length of a terminal branch may be approximately 10 mm from the hypoglossal nerve to the genioglossal or geniohyoid muscles. In some examples, there may be approximately an average of 1.5 terminal branches with a standard deviation of ±0.7 for the right geniohyoid muscle, an average of 4.8 terminal branches with a standard deviation of ±1.4 for the right genioglossus muscle, an average of 2.0 terminal branches with a standard deviation of ±0.9 for the left geniohyoid muscle, and an average of 5.1 terminal branches with a standard deviation of ±1.9 for the left genioglossus muscle.

There may be possible advantages with stimulating at motor points 208A, 208B, 210A, or 210B, as compared to some other techniques. For instance, some techniques utilize cuff electrodes or stimulate at the hypoglossal nerve. Due to the different bifurcation patterns, placing a cuff electrode around the hypoglossal nerve, or generally attaching an electrode to the hypoglossal nerve can be challenging. Also, where cuff electrodes or electrodes that attach to the hypoglossal nerve are used, implanting electrodes around or at each of the hypoglossal nerves requires multiple surgical entry points to attached to both hypoglossal nerves. Moreover, utilizing cuff electrodes or electrodes that attach to the hypoglossal nerves can possibly negatively impact the nerve by tugging, stretching, or otherwise causing irritation. Accordingly, utilizing lead 106 and electrodes 117 that are implanted proximate to the motor points may be beneficial (e.g., less surgery to implant and less impact on the nerve) as compared to techniques where cuff electrodes or electrodes implanted on the hypoglossal nerve are utilized.

Furthermore, stimulating at motor points 208A, 208B, 210A, and/or 210B, such as at the bifurcation point of a motor neuron that attach to muscle fibers, may provide advantages such as for better control of muscle movement. Because motor points 208A, 208B, 210A, and 210B are spatially distributed, by stimulating motor points 208A, 208B, 210A, and/or 210B, the amount of the genioglossus and geniohyoid muscle that is being stimulated can be controlled. Also, stimulating at motor points 208A, 208B, 210A, and/or 210B may allow for more gentle muscle activation. For instance, when stimulation is provided near the trunk of the hypoglossal nerve, even stimulation signal with relatively small amplitude can cause the genioglossus and/or geniohyoid muscle to fully protrude (e.g., there is high loop gain where small stimulation amplitudes cause large muscle protrusion). Fine tuning of how much to protrude the genioglossus and/or geniohyoid muscle may not be available when stimulating at a trunk of the hypoglossal nerve. However, there may be lower loop gain stimulating at motor points 208A, 208B, 210A, and/or 210B. For instance, a stimulation signal having a lower amplitude may move cause the genioglossus and/or geniohyoid muscle to protrude a small amount, and a stimulation signal having a higher amplitude may move cause the genioglossus and/or geniohyoid muscle to protrude a higher amount when stimulating at motor points 208A, 208B, 210A and/or 210B.

The following are example locations of motor points 208A, 208B, 210A, and 210B relative to the midline (x-axis), posterior symphysis 202 (y-axis), and depth (z-axis), where the depth is from the plane formed by the inferior border of symphysis 202 and anterior border of hyoid bone 204.

Motor points 208A may be for the right genioglossus muscle and may be located at 13.48 mm±3.59 from the x-axis, 31.01 mm±6.96 from the y-axis, and 22.58 mm±3.74 from the z-axis. Motor points 210A may be for the right geniohyoid muscle and may be located at 11.74 mm±3.05 from the x-axis, 41.81 mm±6.44 from the y-axis, and 16.29 mm±3.40 from the z-axis. Motor points 208B may be for the left genioglossus muscle and may be located at 9.96 mm±2.24 from the x-axis, 29.62 mm±9.25 from the y-axis, and 21.11 mm±4.10 from the z-axis. Motor points 210B may be for the left geniobyoid muscle and may be located at 11.45 mm±1.65 from the x-axis, 39.63 mm±8.03 from the y-axis, and 15.09 mm±2.41 from the z-axis.

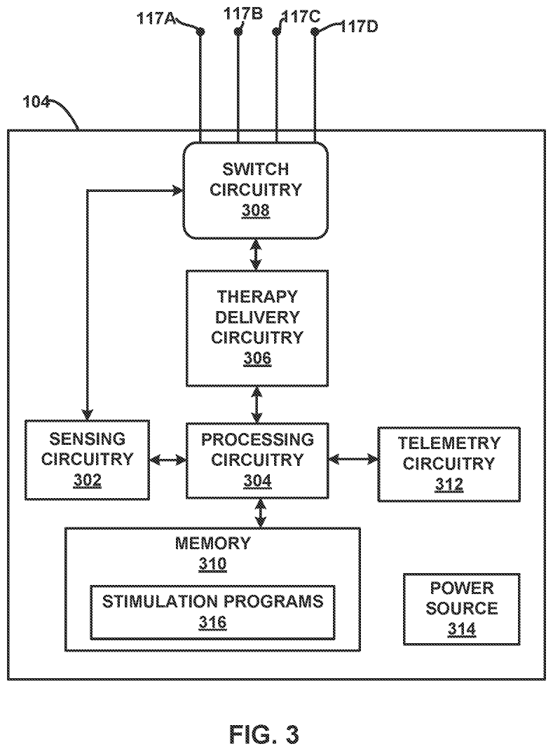

FIG. 3 is block diagram illustrating example configurations of implantable medical devices (IMDs) which may be utilized in the system of FIG. 1. As shown in FIG. 3, IMD 104 includes sensing circuitry 302, processing circuitry 304, therapy delivery circuitry 306, switch circuitry 308, memory 310, telemetry circuitry 312, and power source 314. IMD 104 may include a greater or fewer number of components. For example, in some examples, such as examples in which IMD 104 deliver the electrical stimulation in an open-loop manner, IMD 104 may not include sensing circuitry 302.

Switch circuitry 308 may be configured to, in response to instructions from processing circuitry 304, switch the coupling of electrodes 117 between sensing circuitry 302 and therapy delivery circuitry 306. In examples where sensing circuitry 302 is not used, switch circuitry 308 may not be needed. However, even in examples where sensing circuitry 302 is not used, IMD 104 may include switch circuitry 308 such as to disconnect electrodes 117 from therapy delivery circuitry 306.

In some examples, therapy delivery circuitry 306 may include a plurality of regulated current sources or sinks, with each current source or sink coupled to one of electrodes 117. In such examples, therapy delivery circuitry 306 may control each current source or sink and switching between electrodes 117 may not be necessary for therapy delivery since each one of electrodes 117 is individually controllable.

Although not shown in FIG. 3, in some examples, IMD 104 may include one or more sensors configured to sense posture or position of patient 102. For example, IMD 104 may include accelerometer to determine if patient 102 is lying down. Another example of the one or more sensors is a motion sensor, and movement sensed by the motion sensor may indicate if patient 102 is having restless sleep, which may be indicative of the onset of OSA. Additional examples of the sensors include acoustical sensors or a microphone for detecting vibrations in upper airway 124. Vibrations in upper airway 124 may be indicative of the onset of OSA. In some examples, processing circuity 304 may control delivery of therapy based on information received from the one or more sensors, such as delivery of therapy after sensing an onset of OSA.

In some examples, electrodes 117 may be configured to sense electromyogram (EMG) signals. Sensing circuitry 302 may be switchably coupled to electrodes 117 via switch circuitry 308 to be used as EMG sensing electrodes with electrodes 117 are not being used for stimulation. EMG signals may be used by processing circuitry 304 to detect sleep state and/or low tonal state of protrusor muscles 120 and/or 122 for use in delivering electrical stimulation. In some examples, rather than using electrodes 117 or in addition to using electrodes 117, there may be other electrodes or sensors used to sense EMG signals.

In general, IMD 104 may comprise any suitable arrangement of hardware, alone or in combination with software and/or firmware, to perform the techniques attributed to IMD 104 and processing circuitry 304, therapy delivery circuitry 306, and telemetry circuitry 312 of IMD 104. In various examples, IMD 104 may include one or more processors, such as one or more microprocessors, digital signal processors (DSPs), application specific integrated circuits (ASICs), field programmable gate arrays (FPGAs), or any other equivalent integrated or discrete logic circuitry, as well as any combinations of such components.

The various units of IMD 104 may be implemented as fixed-function circuits, programmable circuits, or a combination thereof. Fixed-function circuits refer to circuits that provide particular functionality and are preset on the operations that can be performed. Programmable circuits refer to circuits that can be programmed to perform various tasks, and provide flexible functionality in the operations that can be performed. For instance, programmable circuits may execute software or firmware that cause the programmable circuits to operate in the manner defined by instructions of the software or firmware. Fixed-function circuits may execute software instructions (e.g., to receive parameters or output parameters), but the types of operations that the fixed-function circuits perform are generally immutable. In some examples, one or more of the units may be distinct circuit blocks (fixed-function or programmable), and in some examples, one or more of the units may be integrated circuits.

IMD 104 may include arithmetic logic units (ALUs), elementary function units (EFUs), digital circuits, analog circuits, and/or programmable cores, formed from programmable circuits. In examples where the operations of IMD 104 are performed using software executed by the programmable circuits, memory 310 may store the instructions (e.g., object code) of the software that processing circuitry 304 receives and executes, or another memory within IMD 104 (not shown) may store such instructions.

IMD 104 also, in various examples, may include a memory 310, such as random access memory (RAM), read only memory (ROM), programmable read only memory (PROM), erasable programmable read only memory (EPROM), electronically erasable programmable read only memory (EEPROM), flash memory, comprising executable instructions for causing the one or more processors to perform the actions attributed to them. Moreover, although sensing circuitry 302, processing circuitry 304, therapy delivery circuitry 306, switch circuitry 308, and telemetry circuitry 312 are described as separate circuitry, in some examples, sensing circuitry 302, processing circuitry 304, therapy delivery circuitry 306, switch circuitry 308, and telemetry circuitry 306 are functionally integrated. In some examples, sensing circuitry 302, processing circuitry 304, therapy delivery circuitry 306, switch circuitry 308, and telemetry circuitry 312 correspond to individual hardware units, such as ASICs, DSPs, FPGAs, or other hardware units.

Memory 310 stores stimulation programs 316 (also called therapy programs 316) that specify stimulation parameter values for the electrical stimulation provided by IMD 104. Memory 310 may also store instructions for execution by processing circuitry 304, in addition to stimulation programs 316. Information related to sensed parameters of patient 102 (e.g., from sensing circuitry 302 or the one or more sensors of IMD 104) may be recorded for long-term storage and retrieval by a user, and/or used by processing circuitry 304 for adjustment of stimulation parameters (e.g., amplitude, pulse width, and pulse rate). In some examples, memory 310 includes separate memories for storing instructions, electrical signal information, and stimulation programs 316. In some examples, processing circuitry 304 may select new stimulation parameters for a stimulation program 316 or new stimulation program from stimulation programs 316 to use in the delivery of the electrical stimulation based on patient input and/or monitored physiological states after termination of the electrical stimulation.

Generally, therapy delivery circuitry 306 generates and delivers electrical stimulation under the control of processing circuitry 304. In some examples, processing circuitry 304 controls therapy delivery circuitry 306 by accessing memory 310 to selectively access and load at least one of stimulation programs 316 to therapy delivery circuitry 306. For example, in operation, processing circuitry 304 may access memory 60 to load one of stimulation programs 316 to therapy delivery circuitry 306.

By way of example, processing circuitry 304 may access memory 310 to load one of stimulation programs 316 to control therapy delivery circuitry 306 for delivering the electrical stimulation to patient 102. A clinician or patient 102 may select a particular one of stimulation programs 316 from a list using a programming device, such as a patient programmer or a clinician programmer. Processing circuitry 304 may receive the selection via telemetry circuitry 312. Therapy delivery circuitry 306 delivers the electrical stimulation to patient 102 according to the selected program for an extended period of time, such as minutes or hours while patient 102 is asleep (e.g., as determined from the one or more sensors and/or sensing circuitry 302). For example, processing circuitry 304 may control switch circuitry 308 to couple electrodes 117 to therapy delivery circuitry 306.

Therapy delivery circuitry 306 delivers electrical stimulation according to stimulation parameters. In some examples, therapy delivery circuitry 306 delivers electrical stimulation in the form of electrical pulses. In such examples, relevant stimulation parameters may include a voltage or current pulse amplitude, a pulse rate, a pulse width, a duty cycle, and/or the combination of electrodes 117 that therapy delivery circuitry 306 uses to deliver the stimulation signal. In some examples, therapy delivery circuitry 306 delivers electrical stimulation in the form of continuous waveforms. In such examples, relevant stimulation parameters may include a voltage or current amplitude, a frequency, a shape of the stimulation signal, a duty cycle of the stimulation signal, or the combination of electrodes 117 therapy delivery circuitry 306 uses to deliver the stimulation signal.

In some examples, the stimulation parameters for the stimulation programs 316 may be selected to cause protrusor muscles 120 and/or 122 to an advanced state (e.g., to open-up airway 124). An example range of stimulation parameters for the electrical stimulation that are likely to be effective in treating OSA (e.g., upon application to the hypoglossal nerves to cause protrusor muscles 120, 122 to protrude or upon application to motor points such as motor points 208A, 208B, 210A, and 210B), are as follows:
a. Frequency or pulse rate: between about 20 Hz and about 50 Hz, and possibly lower such as 2 Hz and 4 Hz. In some examples, the minimum target frequency is used which can achieve muscle tetany (e.g., constant contraction) and provide the required force to open the airway.
b. Current Amplitude: between about 0.1 milliamps (mA) and about 20 mA, and more generally from 0.5 mA to 3 mA, and approximately 1.5 mA.
c. Pulse Width: between about 100 microseconds (μs) and about 500 μs. In some examples, a pulse width of 150 μs might be used for reduced power consumption. In some particular examples, the pulse width is approximately 240 μs. In some cases, shorter pulse widths may be used in conjunction with higher current or voltage amplitudes.

Processing circuitry 304 may select stimulation programs 316 for alternating delivery of electrical stimulation between stimulating the left protrusor muscles 120 and/or 122 and the right protrusor muscles 120 and/or 122 on a time basis, such as in examples where two needles and two leads 106 are implanted. In some examples, there may be some overlap in the delivery of electrical stimulation such that for some of amount of time both left and right protrusor muscles 120 and/or 122 are being stimulated. In some examples, there may be a pause in alternating stimulation (e.g., stimulate left protrusor muscles, a time period with no stimulation, then stimulate right protrusor muscles, and so forth). Processing circuitry 304 may also select stimulation programs 316 that select between different combinations of electrodes 117 for stimulating, such as to stimulate different locations of the hypoglossal nerve(s), which may help with fatigue as well as provide more granular control of how much to protrude tongue 118.

In the example of FIG. 3, therapy delivery circuitry 306 drives electrodes 117 of lead 106. Specifically, therapy delivery circuitry 306 delivers electrical stimulation (e.g., regulated current or voltage pulses at pulse rates and pulse widths described above) to tissue of patient 102 via selected electrodes 117A-117D carried by lead 106. A proximal end of lead 106 extends from the housing of IMD 104 and a distal end of lead 106 extends to a target therapy site, e.g., through inner lumen of the needle. Target therapy sites may include one or both hypoglossal nerves and/or motor points 208A, 210A, 208B and/or 210B. Therapy delivery circuitry 306 may deliver electrical stimulation with electrodes on more than one lead and each of the leads may carry one or more electrodes, such as when patient 102 is implanted with two needles and two leads 106 in tongue 118 for stimulating both hypoglossal nerves simultaneously or bilaterally (e.g., one after the other) or both motor points 208A and 208B and/or motor points 210A and 210B. The leads may be configured as an axial lead with ring electrodes or segmented electrodes and/or paddle leads with electrode pads arranged in a two-dimensional array. The electrodes may operate in a bipolar or multi-polar configuration with other electrodes, or may operate in a unipolar configuration referenced to an electrode carried by the device housing or “can” of IMD 104.

In some examples, processing circuitry 304 may control therapy delivery circuitry 306 to deliver or terminate the electrical stimulation based on patient input received via telemetry circuitry 312. Telemetry circuitry 312 includes any suitable hardware, firmware, software, or any combination thereof for communicating with another device, such as an external programmer. Under the control of processing circuitry 304, telemetry circuitry 312 may receive downlink telemetry (e.g., patient input) from and send uplink telemetry (e.g., an alert) to a programmer with the aid of an antenna, which may be internal and/or external. Processing circuitry 304 may provide the data to be uplinked to the programmer and the control signals for telemetry circuitry 312 and receive data from telemetry circuitry 312.

Generally, processing circuitry 304 controls telemetry circuitry 312 to exchange information with a medical device programmer and/or another device external to IMD 104. Processing circuitry 304 may transmit operational information and receive stimulation programs or stimulation parameter adjustments via telemetry circuitry 312. Also, in some examples, IMD 104 may communicate with other implanted devices, such as stimulators, control devices, or sensors, via telemetry circuitry 312.

Power source 314 delivers operating power to the components of IMD 104. Power source 314 may include a battery and a power generation circuit to produce the operating power. In some examples, the battery may be rechargeable to allow extended operation. Recharging may be accomplished through proximal inductive interaction between an external charger and an inductive charging coil within IMD 104. In other examples, an external inductive power supply may transcutaneously power IMD 104 whenever electrical stimulation is to occur.

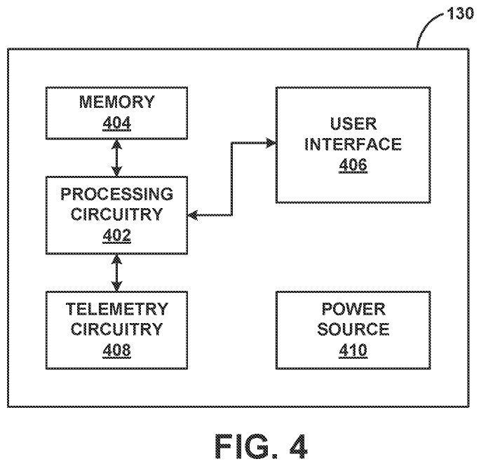

FIG. 4 is a block diagram illustrating an example configuration of an external programmer 130. While programmer 130 may generally be described as a hand-held computing device, programmer 130 may be a notebook computer, a cell phone, or a workstation, for example. As illustrated in FIG. 4, external programmer 130 may include processing circuitry 402, memory 404, user interface 406, telemetry circuitry 408, and power source 410.

In general, programmer 130 comprises any suitable arrangement of hardware, alone or in combination with software and/or firmware, to perform the techniques attributed to programmer 130, and processing circuitry 402, user interface 406, and telemetry module 408 of programmer 130. Examples of processing circuitry 402 may include one or more processors, such as one or more microprocessors, DSPs, ASICs, FPGAs, or any other equivalent integrated or discrete logic circuitry, as well as any combinations of such components. Examples of memory 404 may include RAM, ROM, PROM, EPROM, EEPROM, flash memory, a hard disk, a CD-ROM, comprising executable instructions for causing the one or more processors to perform the actions attributed to them. Moreover, although processing circuitry 402 and telemetry circuitry 408 are described as separate circuitry, in some examples, processing circuitry 402 and telemetry circuitry 408 are functionally integrated. In some examples, processing circuitry 402 and telemetry circuitry 408 correspond to individual hardware units, such as ASICs, DSPs, FPGAs, or other hardware units.

In some examples, memory 404 may further include program information (e.g., stimulation programs) defining the electrical stimulation, similar to those stored in memory 310 of IMD 104. The stimulation programs stored in memory 404 may be downloaded into memory 310 of IMD 104.

User interface 406 may include a button or keypad, lights, a speaker for voice commands, a display, such as a liquid crystal (LCD), light-emitting diode (LED), or cathode ray tube (CRT). In some examples the display may be a touch screen. As discussed in this disclosure, processing circuitry 402 may present and receive information relating to electrical stimulation and resulting therapeutic effects via user interface 406. For example, processing circuitry 402 may receive patient input via user interface 406. The input may be, for example, in the form of pressing a button on a keypad or selecting an icon from a touch screen.

Processing circuitry 402 may also present information to the patient in the form of alerts related to delivery of the electrical stimulation to patient 102 or a caregiver via user interface 406. Although not shown, programmer 130 may additionally or alternatively include a data or network interface to another computing device, to facilitate communication with the other device, and presentation of information relating to the electrical stimulation and therapeutic effects after termination of the electrical stimulation via the other device.

Telemetry circuitry 408 supports wireless communication between IMD 104 and programmer 130 under the control of processing circuitry 402. Telemetry circuitry 408 may also be configured to communicate with another computing device via wireless communication techniques, or direct communication through a wired connection. In some examples, telemetry circuitry 78 may be substantially similar to telemetry circuitry 312 of IMD 104 described above, providing wireless communication via an RF or proximal inductive medium. In some examples, telemetry circuitry 408 may include an antenna, which may take on a variety of forms, such as an internal or external antenna.

Examples of local wireless communication techniques that may be employed to facilitate communication between programmer 130 and another computing device include RF communication according to the 802.11 or Bluetooth specification sets, infrared communication (e.g., according to the IrDA standard), or other standard or proprietary telemetry protocols. In this manner, other external devices may be capable of communicating with programmer 130 without needing to establish a secure wireless connection.

Power source 410 delivers operating power to the components of programmer 130. Power source 410 may include a battery and a power generation circuit to produce the operating power. In some examples, the battery may be rechargeable to allow extended operation.

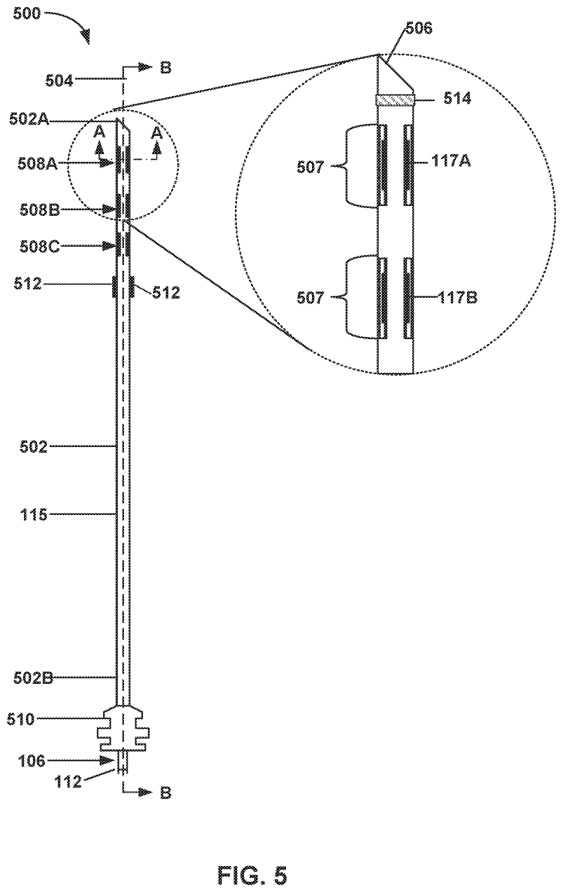

FIG. 5 is a conceptual diagram illustrating an example needle assembly 500 for system 100 of FIG. 1. Example needle assembly 500 may include needle 115 and lead 106 of system 100. As illustrated in FIG. 5, an elongated lead body 112 of lead 106 may be disposed within an inner lumen of needle 115 at a plurality of positions. In one such position, as illustrated in FIG. 5, one or more electrodes 117A disposed on lead body 112 may corresponding to one or more openings 507 on elongated needle body 502 of needle 115. In other positions, one or more electrodes 117 may be at a different longitudinal position along longitudinal axis 504 and/or a different radial orientation than one or more openings 507.

Needle 115 may include an elongated needle body 502 which may include a distal portion 502A and a proximal portion 502B. Elongated needle body 502 may have an annular shape around longitudinal axis 504 and may define an inner lumen (not pictured).

Proximal portion 502B may include an attachment member 510 disposed at a proximal end of proximal portion 502B. Attachment member 510 may provide an entrance into the inner lumen of elongated body 502. A surgeon may insert lead body 112 of lead 106 into the inner lumen through attachment member 510. In some examples, during insertion of needle 115 into tissue of patient 102, the surgeon may insert a trocar or other similar device into the inner lumen of needle 115 through attachment member 510. Once needle 115 is inserted, the surgeon may remove the trocar from attachment member 510 and insert lead body 112.

In some examples, attachment member 510 may include one or more reference marks. The surgeon may align the one or more reference marks on attachment member 510 with one or more reference marks on lead body 112 to position and orient one or more electrodes 117 with one or more openings 507 of needle 115. In some examples where proximal portion 502B does not include attachment member 510, needle 115 may include one or more reference marks disposed on the proximal end of elongated needle body 502.

Distal portion 502A may include one or more openings 507, a puncturing tip 506, and one or more sensing electrodes 512. Distal portion 502A may include one or more sets of openings 508A-D (hereinafter referred to as “sets of openings 508”). The one or more sets of openings 508 may be positioned at different longitudinal positions along elongated needle body 502. For example, the set of openings 508B may be positioned proximally to the set of openings 508A along elongated needle body 502. In some examples, the distances between two adjacent sets of openings 508 may correspond to the distance between the corresponding sets of electrodes 117 of lead 106. In some examples, as illustrated in FIG. 5, needle 115 may include three sets of openings 508 (sets of openings 508A-C). In other examples, needle 115 may include one, two, or four or more sets of openings 508. Each of the sets of openings 508 may include one or more openings 507 arranged around the circumference of needle 115.

Puncturing tip 506 is disposed on the distal end of needle 115 and is configured to puncture tissue of patient 102 as needle 115 is navigated to the target treatment area. In some examples, distal portion 502A may be configured to dilate a puncture created by puncturing tip 506, e.g., via a gradual increase in the outer diameter of distal portion 502A along longitudinal axis 504. In some examples, distal portion 502A and/or puncturing tip 506 may further include a radiopaque marker 514 configured to allow the surgeon to locate the position of distal portion 502A of needle 115 within patient 102 during implantation.

In some examples, distal portion 502A may include one or more sensing electrodes 512 disposed on elongated needle body 502. One or more sensing electrodes 512 may be configured to detect electrical signals in tissue of patient 102 and to transmit the detected electrical signals to a medical device (e.g., IMD 104). One or more sensing electrodes 512 may be connected to the medical device via one or more conductors (not pictured) disposed along the outer surface of elongated needle body 502. In some examples, as illustrated in FIG. 5, one or more sensing electrodes 512 may be positioned at a different axial position than one or more openings 507 relative to longitudinal axis 504. In other examples, one or more sensing electrodes 512 may have a same axial position as one or more sets of openings 508. For example, sensing electrodes 512 may be disposed between one or more openings 507 of the set of openings 508A, between one or more openings 507 of the set of openings 508B, or the like.

In some examples, lead body 112 may include one or more fixation members (not pictured) configured to fix lead 106 within tissue of patient 102. The fixation member may include multiple sets of tines which engage the surrounding tissue when a distal portion of lead 106 (e.g., lead distal end 116) is positioned at the target therapy delivery site. The tines of fixation member may extend radially outward and proximally at an angle relative to longitudinal axis 504 of lead body 112 to prevent or reduce retraction of lead body 112. For instances, the tines may include springs that in an uncompressed state extend the tines outwards. Tines of the fixation member may be collapsible against lead body 112 when lead 106 is held within the inner lumen of elongated needle body 502. Upon removal of needle 115, the tines of the fixation member may spread to a normally extended position (e.g., due to the spring bias) to engage with surround tissue and resist proximal and lateral migration of lead body 112. For instance, the tines may be normally biased to the extended position but retracted against elongated needle body 502 for implantation. When needle 115 is removed, the tines extend outward to their uncompressed state. In some examples, the fixation member may additionally or alternatively include one or more hooks, barbs, helices, or other fixation mechanisms extending from one or more longitudinal locations along lead body 112 and/or lead distal end 116.

In some examples, needle assembly 500 may include a sheath (not pictured) disposed within an inner lumen of elongated needle body 502 and around lead 106. The sheath may constrain the one or more fixation members (e.g., tines) in a collapsed configuration against the body of lead 106 and prevent the one or more fixation members from catching on needle 115 (e.g., on openings 507) as lead 106 is advanced, retracted, and/or rotated within elongated needle body 502.

The sheath may include one or more openings corresponding to electrodes 117 on lead 106 and/or openings 507 on elongated needle body 502. In some examples, the sheath includes one or more electrically insulative materials and electrically insulates at least portions of lead 106 from elongated needle body 502.

In some examples clinician may deliver test stimulation signals to protrusor muscles 120 and/or 122 via needle 115. Based on a determination that the placement of needle 115 is accurate, the clinician may advance lead 106 with the sheath into the inner lumen of needle 115. The clinician may then confirm the accuracy of the placement of needle 115 by delivering test stimulation signals to protrusor muscles 120 and/or 122 via lead 106 and observing the results. Based on a confirmation of the placement of needle 115, the clinician may retract needle 115 while leaving lead 106 and the sheath in place.

In some examples, rather than first delivering test stimulation signals to protrusor muscles 120 and/or 122 via needle 115, the clinician may advance lead 160 with the sheath into the inner lumen of needle 115, and deliver test stimulation signals to protrusor muscles 120 and/or 122 via lead 106 (e.g., and through the one or more openings in the sheath).

Once needle 115 is retracted, the clinician may deliver additional test stimulation signals and/or stimulation signals, e.g., to determine the efficacy of the stimulation signals. The clinician may retract the sheath from around lead 106 and cause the one or more fixation members to extend radially outwards of lead 106. The one or more fixation members may then affix lead 106 within the tissue of the patient.

Needle assembly 500 may operate in a unipolar configuration (e.g., a unipolar needle electrode) to stimulate hypoglossal nerve(s) and/or motor points (e.g., one or more of motor points 208A, 208B, 210A, or 210B) in the tongue of a patient. For example, a surgeon may guide distal end 502A of elongated body 502 to a location proximal to the hypoglossal nerve and/or the motor point (e.g., one or more of motor points 208A, 208B, 210A, or 210B) and deliver stimulation signals through one or more electrodes 117 of lead 106 to the hypoglossal nerve and/or the motor point. In such cases, electrodes 117 are coupled to a return electrode (e.g., a ground pad), where the return electrode is secured to the patient's skin. In some examples, electrodes 117 may be electrically isolated from another, and in such examples, one of electrodes 117 may be used to output the stimulation signals and another of electrodes 117 may provide the return path. In some examples, one or more electrodes may be positioned on the outer surface of elongated body 502 and may be configured to deliver stimulation signals to hypoglossal nerve(s) and/or motor points to determine an optimal placement location for lead 106.

The stimulation signals delivered to the hypoglossal nerve and/or the motor point (e.g., one or more of motor points 208A, 208B, 210A, or 210B) may cause a muscle contraction of a protrusor muscle, which may generate electrical signals. The electrical signals generated during the muscle contraction may be detected by electrodes 117 and/or sensing electrodes 512. The surgeon may control the medical device to receive the electrical signals and output information indicative of the one or more electrical signals on a display device. The surgeon may then determine a target treatment site based on the output information. In some examples, reception of electrical signals may not be necessary and visual inspection to determine if tongue 118 protruded or if protrusor muscles 120 and/or 122 activated may be sufficient. Based on the determination, the surgeon may reposition needle assembly 500 within tissue of patient 102 such that needle assembly 500 is positioned at the target treatment site.

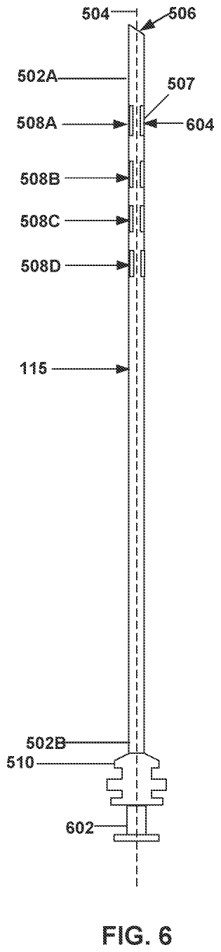

FIG. 6 is a conceptual diagram illustrating an example needle 115 of needle assembly 500 of FIG. 5. Needle 115 includes elongated body 502 defining an inner lumen 604. Proximal portion 502B may include attachment member 510 disposed at a proximal end of proximal portion 502B. In some examples, as illustrated in FIGS. 5 and 6, inner lumen 604 of needle 115 may be configured to accept lead 106 and/or trocar 602. A surgeon may insert trocar 602 into inner lumen 604 of needle 115 during insertion of needle 115 into tissue of patient 102 to facilitate the removal of excess bodily fluids (e.g., blood) at the implantation site of needle 115.

Inner lumen 604 may have an inner diameter large enough to receive a lead (e.g., lead 106) used for OSA therapy. In some examples, inner lumen 604 may have an inner diameter of between about 0.03 inches (in) and about 0.17 in. In some examples inner lumen 604 has an inner diameter of between about 0.038 in. and about 0.154 in. In some examples, elongated body 502 may have a wall size between inner lumen 604 and outer surface of elongated body 502 of between about 0.003 in. to about 0.015 in. In some examples, elongated body 502 has a wall size of between about 0.003 in. and about 0.012 in. In some examples, inner lumen 604 may have a continuous diameter from distal portion 502A to proximal portion 502B. In other examples, at least a portion of inner lumen 604 may have a larger/smaller inner diameter than another portion of inner lumen 604. For example, a proximal portion of inner lumen 604 at proximal portion 502B of elongated body 502 may have a relatively larger inner diameter to facilitate the insertion of lead 14 and/or trocar 602 into inner lumen 604.

Elongated body 502 may have a continuous outer diameter from distal portion 502A to proximal portion 502B. In other examples, the outer diameter of elongated body 502 may increase along longitudinal axis 504 from distal portion 502A to proximal portion 502B. The increase in the outer diameter of elongated body 502 may be continuous or discrete. Elongated body 502 may have an increasing outer diameter to facilitate the dilation of an orifice created by puncturing tip 506 of needle 115 during the implantation process. In some examples, elongated body 502 may have a needle gauge size of about 12 gauge or greater. In some examples elongated body 502 may have an outer diameter of about 14 gauge or greater.

Elongated body 502 of needle 115 may include electrically conductive portion(s) and electrically insulative portion(s). In some examples, needle 115 may include an electrically insulative segment of elongated body 502 attached to an electrically conductive segment of elongated body 502. In other examples, elongated body 502 may be made entire of an electrically conductive material and an electrically insulative coating may be applied over at least a portion of elongated body 502 to form the electrically insulative portion(s).

The electrically insulative portion(s) of needle 115 may facilitate the delivery of stimulation signals to tissue of patient 102 through electrodes 117 of lead 106 by controlling the area of needle 115 through which electrodes 117 may transmit the electrical stimulation signals. The electrically insulative portion(s) of needle 115 may include portions of the outer surface of elongated body 502 and/or inner lumen 604 around one or more openings 508. The electrically insulative material may be disposed on the portions of the outer surface of elongated body 502 to reduce electrical shunting away from the target treatment site. In some examples, the electrically insulative portion includes the entire inner lumen 604. The electrically insulative material may be disposed on inner lumen 604 to avoid an electrical short circuit of one or more of electrodes 117. The electrically insulative material forming the electrically insulative portion(s) may include any suitable non-conducting material, including, but are not limited to, biocompatible polymers, parylene, vinyl, silicone, vinyl-silicone, polyurethane, or a composite of aluminum oxide/boron nitride (AOBN), polyvinylidene fluoride, polyethylene, polypropylene, polydimethylsiloxane, perylene, polyamide, polytetrafluoroethylene, polymethylmethacrylate, polyimide, polyurethane, liquid crystalline polymers, nanocomposites, or the like.

The electrically conductive portion(s) of needle 115 may include electrically conductive materials positioned on a distal portion (e.g., distal portion 502A) of elongated body 502. In some examples, the electrically conductive portion(s) of needle 115 may transmit electrical signals between electrodes (e.g., electrodes 117, sensing electrodes 512, or the like) and IMD 104. In some examples, electrodes (e.g., sensing electrodes 512) may be attached to the electrically conductive portion(s) of needle 115. In some examples, the electrically conductive portion(s) of needle 115 may include, but are not limited to, puncturing tip 506, portions of elongated body 502 not covered by the electrically insulative material, and/or proximal portion 502B. The electrically conductive material may include, but is not limited to, stainless steel, cobalt-chrome alloy, titanium, nickel-titanium alloy (nitinol), gold, platinum, silver, iridium, tantalum, tungsten, or the like.

Distal portion 502A of needle 115 includes one or more openings 507. Each of the one or more openings 507 may extend from the outer surface of elongated body 502 to inner lumen 604. The one or more openings 507 may be arranged into sets of openings 508 arranged along longitudinal axis 504. In some examples, the distance between the proximal-most set of openings 508 and the distal-most set of openings 508 may be between about 20 mm and about 30 mm. In some examples, each of the sets of openings 508 may have one or more openings 507 arrange circumferentially around elongated body 502. For example, each of the sets of openings 508 may include one opening 507, two openings 507, or three or more openings 507.

In some examples, each of the sets of openings 508 may have the same number of openings 507 at the same orientations. In other examples, each of the sets of openings 508 may have a different number of openings 507 and/or have openings 507 at different orientations. For example, the set of openings 508A may have four openings 507 arranged 90 degrees apart and the set of openings 508B may have six openings 507 arranged 60 degrees apart.

Openings 507 may have, but are not limited to, a rectangular, circular, oval, trapezoidal, or a square shape. In some examples, each of openings 507 of each of the sets of openings 508 may extend less than 180 degrees around the circumference of elongated body 502. In some examples openings 507 may correspond to electrodes 117 of lead 106.

Openings 507 may be formed by any suitable technique. In some examples, openings 507 are formed by a mechanical technique, such as, but not limited to, laser cutting, drilling, or punching of elongated body 502. In other examples, openings 507 are formed by a chemical technique, such, but not limited to, the selective dissolution of one or more sections of elongated body 502, or any combination thereof. In some examples the electrically insulative material may be applied to some portions of elongated body 502 prior to the formation of openings 507.

An opening 507 may correspond to an electrode 117 when opening 507 is aligned with electrode 117 such that elongated body 502 of needle 115 does not obscure a path between electrode 117 and the tissue of patient 102. Each of openings 507 may correspond to one or more electrodes 117 on lead 106 such that electrodes 117 may transmit electrical stimulation signals to the tissue of patient 102 and/or detect electrical signals from the tissue of patient 102 through at least some of the one or more openings 507. In some examples, each of openings 507 of needle 115 corresponds to a different electrode 117. In other examples, each of openings 507 may correspond to two or more electrodes 117. In other examples, each electrode 117 may correspond to two or more openings 507.

When lead 106 is in a first position within inner lumen 604, one or more openings 507 may correspond with electrodes 117. When lead 106 is in a second position within lumen 604, electrodes 117 of lead 106 may be completely occluded by elongated body 502. Optionally, lead 106 may be placed in one or more other positions within lumen 604 such the electrodes 117 may partially correspond with openings 507, are at a same axial position along longitudinal axis 504 as openings 507, and/or are at a same orientation as openings 507.

In some examples, trocar 602 includes one or more electrodes (not pictured) disposed on an elongated body of trocar 602. The one or more electrodes disposed on trocar 602 may be configured to deliver test stimulation signals and/or stimulation signals to protrusor muscles 120 and/or 122. In some examples, trocar 602 is disposed within inner lumen 604 of needle 115. Needle 115 may be retracted proximal of trocar 602 and a sheath (not pictured) may be advanced over the elongated body of trocar 602. In some examples, trocar 602 may be disposed within the sheath and may be advanced, along with the sheath, out of a distal end of needle 115.

The sheath may be configured to electrically isolate at least portions of trocar 602 from tissue of the patient. The sheath may include one or more electrically insulative materials, e.g., as described above. The sheath may include one or more openings configured to align with the one or more electrodes disposed on trocar 602 and/or openings 507 on needle 115. The physician may deliver test stimulation signals and/or stimulation signals to protrusor muscles 120 and/or 122 through trocar 602 disposed within the sheath. Based on a determination that the position of the trocar 602 is satisfactory, the physician may retract trocar 602 from the sheath and advance lead 106 into the sheath. The physician may then align electrodes 117 disposed on lead 106 with the one or more openings on the sheath to deliver stimulation signals to protrusor muscles 120 and/or 122.

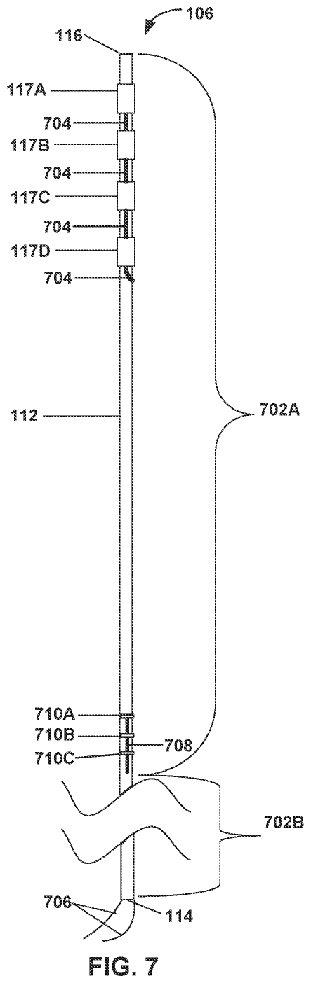

FIG. 7 is a conceptual diagram illustrating implantable lead 106 of needle assembly 500 of FIG. 5. FIG. 7 illustrates lead body 112 of lead 106, lead body 112 having distal portion 702A and proximal portion 702B. Distal portion 702A forms part of lead 106 that is disposed within inner lumen 604 of needle 115 of needle assembly 500. Lead 106 includes one or more electrodes 117, and FIG. 7 illustrates lead 106 with four electrodes 117 A-D (collectively referred to as “electrodes 117) spaced apart longitudinally along lead body 112. In some examples, lead 106 may have one, two, three, or five or more electrodes 117 spaced longitudinally along lead body 112. In some examples, each of electrodes 117 may be a plurality of electrodes at a same axial position relative to longitudinal axis 504 and arranged around the circumference of lead body 112.

Lead body 112 may be a flexible elongated body through which insulated electrical conductors 704 extend to respective electrodes 117. The distal-most electrode (e.g., electrode 117A) may be adjacent or proximate to lead distal end 116. Each of electrodes 117 may be spaced proximally from the respective adjacent one of electrodes 117 by respective interelectrode distances. In some examples, the respective interelectrode distances may be the distance between any two adjacent electrodes 117. In other examples, at least one of the respective interelectrode distance may be different from another of the respective interelectrode distances.

The electrical conductors 704 extend to respective electrodes 117 from proximal contacts 704 at proximal end 114 of lead body 112. In some examples, the electrical conductors 704 may be arranged as a plurality of coils. The plurality of coils may increase the flexibility of lead 106 so that lead 106 can bend at distal end 116. In some examples, the coils may be exposed along the locations of electrodes 117 such that the coils form electrode 117. In some examples, rather than electrodes 117 being pad electrodes or ring electrodes, the coils form coil electrodes and provide additional flexibility.

In some examples, each one of electrodes 117 may have an equivalent electrode length (e.g., longitudinal extend of electrodes 117 along lead body 112). The electrode length may be about 3 mm or less. In some examples, electrodes 117 may have electrode lengths that are different from each other in order (e.g., to optimize placement of electrodes 117 or the resulting electrical field of stimulation relative to targeted stimulation sites corresponding to left and right hypoglossal nerves or branches of hypoglossal nerves and/or motor points of protrusor muscles 120 and/or 122). In some examples, electrodes 117 may have a microscopic coating applied to outer surfaces of electrodes 117 to reduce the impedance of electrodes 117.

In some examples, as illustrated in FIG. 7, interelectrode distances between adjacent electrodes 117 may be substantially equal. In other examples, the interelectrode distances may be different from each other (e.g., in order to optimize placement of electrodes 117 relative to the target stimulation sites). The interelectrode distances may be about 3 mm or less. In some examples, where lead 106 has a bipolar configuration, electrodes 117 may form anode and cathode pairs for delivering bipolar stimulation in portion of the protrusor muscles 120 and/or 122 (e.g., either the left or right protrusor muscles or a proximal and/or distal portion of portion of the protrusor muscles). A second set of electrodes 117 may form a second anode and cathode pair for delivering bipolar stimulation in a different portion of protrusor muscles 120 and/or 122 (e.g., the other of the left or right portions of the other of the proximal or distal portions). Accordingly, the interelectrode spacing between the two bipolar pairs of electrodes 117 may be different than the interelectrode spacing between the anode and cathode within each bipolar pair of electrodes 117.

In some examples, for a unipolar configuration, housing 108 of IMD 104 may include an electrode that functions as a cathode and a part of the anode and cathode pair with one or electrodes 117. In other examples, housing 108 may be an anode.

In some examples, the total distance encompassed by electrodes 117 along distal portion 702A of lead body 112 may be between approximately 20 mm and about 30 mm. In one example, the total distance is between 20 mm and 22 mm. In other examples, the total distance may be shorter.

In some examples, each of electrodes 117 may be a circumferential ring electrode which may be uniform in diameter with lead body 112. As described above, electrodes 117 may include other types of electrodes such as tip electrodes, helical electrodes, coil electrodes, segmented electrodes, button electrodes, or the like. For example, the distal-most electrode 117A may be provided as a tip electrode at lead distal end 116 with the remaining three electrodes 117B-D being ring electrodes. In some examples, when electrode 117A is positioned at distal end 116, electrode 117A may be a helical electrode configured to screw into the muscle tissue at the implant site to additionally serve as a fixation member for anchoring the distal portion 702A of lead 106 at the targeted therapy delivery site. In some examples, one or more of electrodes 117 may be a hook electrode or barbed electrode to provide active fixation of the distal portion 702A of lead 106 at the therapy delivery site.

Lead body 112 may include a plurality of proximal connectors 706 that engage with connector assembly 110 of IMD 104. Accordingly, the length of elongated body 112 from distal portion 502A to lead proximal end 114 may be selected to extend from a target therapy delivery site in protrusor muscles 120 and/or 122 to a location where IMD 104 is implanted. In some examples, IMD 104 may be implanted in a pectoral region of patient 102. The length of lead body 112 may be up to about 10 centimeters (cm) or up to about 20 cm as examples. In some examples, lead body 112 may be about 25 cm or less in length. In other examples, longer or shorter lead body lengths may be used based on anatomy and size of patient 102.

Lead body 112 may have a plurality of reference marks to allow a surgeon to properly align lead 106 in one or more positions within needle 115. The reference marks may be positioned at a proximal end of distal portion 502A and/or a at a distal end of proximal portion 502B to align the reference marks on lead body 112 with one or more reference marks on needle 115. In some examples, as illustrated in FIGS. 6 and 7, the reference marks on lead body 112 may be aligned with a proximal end of needle 115 and/or attachment member 510 of needle 115. The reference marks on lead 106 may include vertical reference marks 708 and/or horizontal reference marks 710A-C (hereinafter referred to as “horizontal reference marks 710”). While the example lead 106 of FIG. 7 illustrates three horizontal reference marks 710, other leads 106 may include one, two, or four or more horizontal reference marks 710. Each of reference marks 708 and/or 710 may correspond to a pre-determined position of lead 106 within needle 115.

A surgeon may use vertical reference marks 708 to determine the orientation of lead 106 and/or electrodes 117 of lead 106 relative to openings 507 of needle 115. For example, one vertical reference mark 708 may indicate that, when aligned with a reference mark on needle 115, electrodes 117 of lead 106 are in a different orientation than openings 507 of needle 115 and are occluded by elongated body 502 of needle 115. Another vertical reference mark 708 may indicate that, when aligned with the reference mark on needle 115, electrodes 117 of lead 106 are in the same orientation as openings 507. Horizontal reference marks 510 may indicate that, when aligned with a reference mark on needle 115, electrodes 117 of lead 106 are fully exposed to tissue of patient 102 through openings 507 of needle 115, partially occluded by elongated body 502 of needle 115, or completely occluded by elongated body 502 of needle 115. In some examples, when lead 106 is aligned with a first reference mark (e.g., reference mark 510A) where electrodes 117 are aligned with openings 507, the distal tip of lead 106 may be flush with a distal end (e.g., puncturing tip 506) of needle 115.

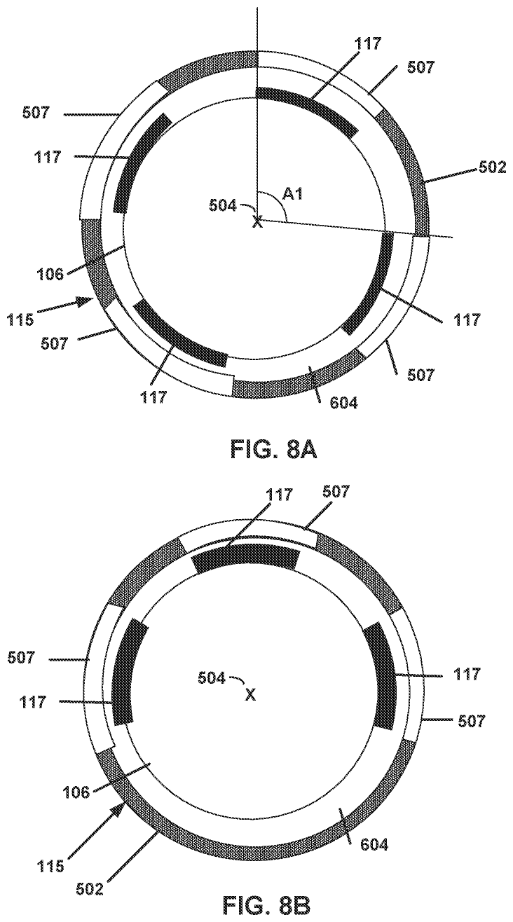

FIGS. 8A-C are conceptual diagrams illustrating cross-sectional views of different example electrode configurations of needle assembly 500 of FIG. 5 taken along line A-A. FIGS. 8A and 8B illustrated electrode configurations with a plurality of electrodes 117 disposed at different orientations around the circumference of lead 106. FIG. 8C illustrates a single electrode 117 (e.g., a ring electrode) disposed around a circumference of lead 106 with a plurality of openings 507 in needle 115.

FIG. 8A illustrates an example electrode configuration with four electrodes 117 arranged equidistant around a circumference of lead 106 and four openings 507 arranged equidistant around a circumference of needle 115. As illustrated in FIG. 8A, the orientation of electrodes 117 and openings 507 relative to each other may be determined based on a reference angle A1 between one electrode 117 and one opening 507. Reference angle A1 may be pre determined based on the size, number, and distribution of electrodes 117 on lead 106 and of openings 507 on needle 115. In some examples, electrodes 117 and openings 507 are considered to be aligned in the correct orientation if reference angle A1 between one of electrodes 117 and one of openings 507 is substantially similar to the pre-determined value of reference angle A1. While the example electrode configuration in FIG. 8A illustrates four equally space electrodes 117 corresponding to four equally spaced openings 507, other example configurations may have one, two, three, or five or more electrodes 117 and/or openings 507. In some examples, in the correct orientation, two or more electrodes 117 of lead 106 may be exposed to tissue of patient 102 through a single opening 507. In some examples, in the correct orientation, a single electrode 17 may be exposed to tissue of patient 102 through two or more adjacent openings 507.

FIG. 8B is a conceptual diagram illustrating a cross-sectional view of another example electrode configuration of needle assembly 500 of FIG. 5 taken along line A-A. FIG. 8B illustrates a biased arrangement of electrodes 117 on lead 106 where electrodes 117 are not disposed on a substantial portion of lead 106. In some examples, the substantial portion may be about 50 percent or less of the circumference of lead 106. In some examples, as illustrated in FIG. 8B, needle 116 may have openings 507 corresponding to electrodes 117 on lead 106 but may not have openings on portions of elongated body 502 corresponding to the substantial portion of lead 106. Needle assembly 500 having a lead 106 with a biased arrangement of electrodes 117 may be positioned within protrusor muscles 120 and/or 122 such that electrodes 117 are oriented towards the hypoglossal nerve(s). FIG. 8C is a conceptual diagram illustrating a cross-sectional view of another example electrode configuration of needle assembly 500 of FIG. 5 taken along line A-A. FIG. 8C illustrates lead 106 having a single electrode 117 disposed around lead 106. Elongated body 502 of needle 115 includes openings 507 that expose portions of electrode 117 to tissue of patient.

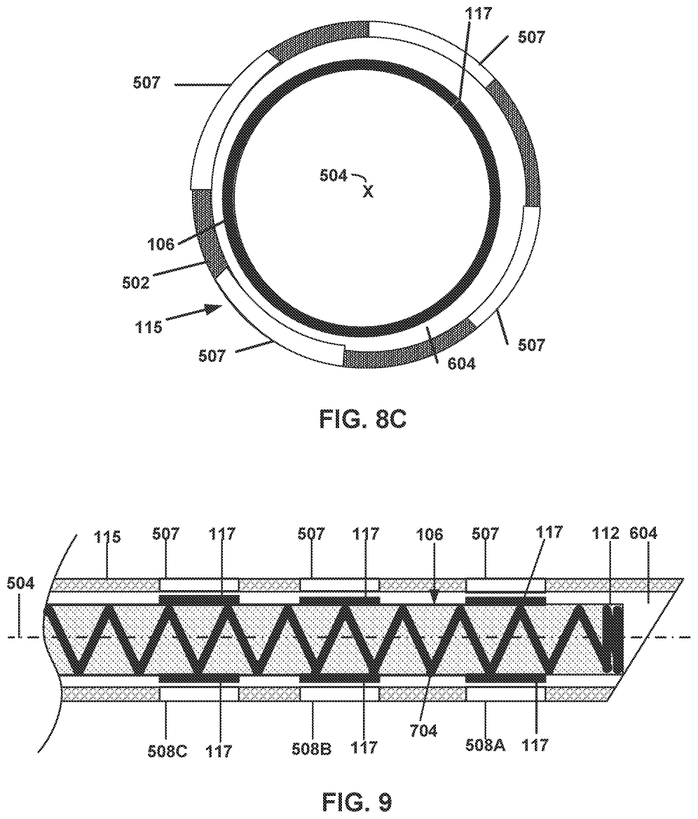

FIG. 9 is a conceptual diagram illustrating a cross-sectional view of needle assembly 500 of FIG. 5 taken along line B-B. As illustrated in FIG. 9, electrical conductors 704 may be arranged in a coil within lead body 112 of lead 106 and electrically connects electrodes 117 with a medical device (not pictured). In other examples, electrical conductors 704 may be arranged in other configurations. For example, electrical conductors 704 may be arranged axially along longitudinal axis 504 of lead 106. In other examples, electrical conductors 704 may disposed on an outer surface of lead 106. When electrical conductors 704 are disposed on the outer surface of lead 106, electrical conductors 704 may have an insulative coating disposed radially outward of electrical conductors 704.

As illustrated in FIG. 9, lead 106 may be manipulated (e.g., advanced, retracted, and/or rotated) within inner lumen 604 of needle 115. The surgeon may manipulate lead 106 within inner lumen 604 to align electrodes 117 of lead 106 with openings 507 of needle 115 such that at least some of electrodes 117 correspond to at least some of openings 507. While FIG. 9 illustrates that electrodes 117 may correspond to openings 507 when the entirety of electrodes 117 are exposed through openings 507, in other examples electrodes 117 may correspond to openings 507 if a sufficient percentage of electrodes 117 are exposed through openings 507 such that an electrical stimulation signal with the proper parameters (e.g., amplitude, frequency, and the like) is delivered to hypoglossal nerve(s) of patient 102.

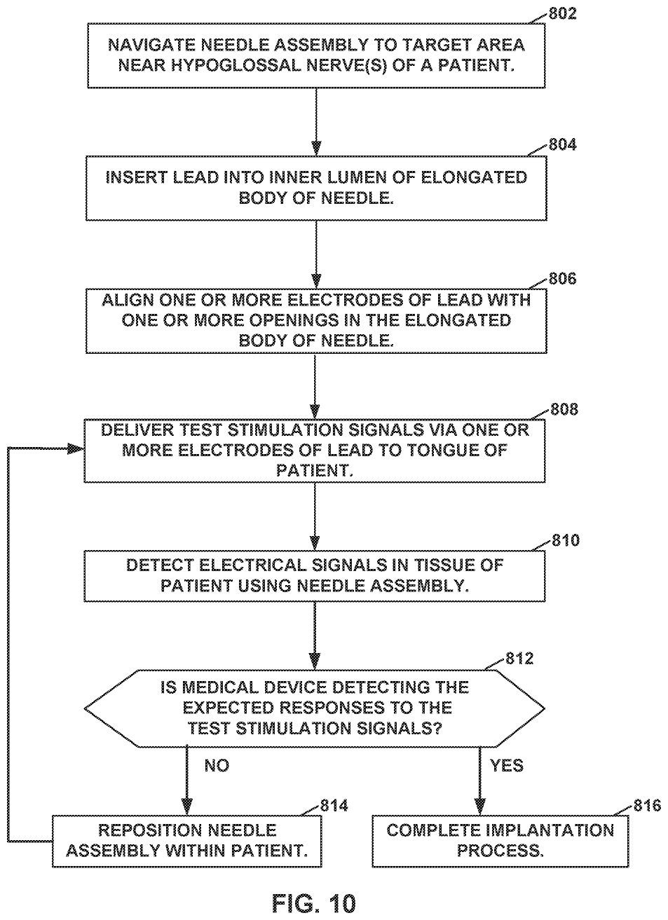

FIG. 10 is a flowchart illustrating an example process of implanting an example needle assembly (e.g., needle assembly 500) near the hypoglossal nerve(s). A medical professional, e.g., a surgeon may navigate needle assembly 500 to a target area near hypoglossal nerve(s) of a patient (802). The surgeon may insert a needle (e.g., needle 115) of needle assembly 500 through tissue near a chin of patient 102 and through tongue 118 of patient 102 to the target area. The target area may be within one or more of protrusor muscles 120 and/or 122 and near one or more hypoglossal nerve(s) and/or motor points (e.g., motor points 208A, 208B, 210A, and/or 210B). Needle 115 may include an elongated body (e.g., elongated body 502) for implantation of needle assembly 500 for treating OSA. In some examples, during insertion of needle 115 into tissue of patient 102 and navigation of needle assembly 500 to areas near hypoglossal nerve(s) of patient 102, the medical professional may insert a trocar (e.g., trocar 602 or a like device) into inner lumen 604 of elongated body 502 of needle 115 to control the amount of bodily fluids (e.g., blood) near needle assembly 500.

The medical professional may insert lead into inner lumen 604 of elongated body 502 of needle 115 (804). In some examples, the medical professional may insert lead 106 into inner lumen 604 of needle 115 after navigating needle assembly 500 to the target area and removing trocar 602 from inner lumen 604 of needle 115. In other examples, the medical professional may insert lead 106 prior to, during, or after navigation of needle assembly 500 to the target area. In some examples, the medical professional dispose lead 106 within inner lumen 604 at a first position until needle 115 is near the hypoglossal nerve(s) and/or motor points of patient 102. At the first position, one or more electrodes 117 on lead 106 may be completely occluded by elongated body 502 of needle 115.

The medical professional may align one or more electrodes 117 of lead 106 with one or more openings 507 in elongated body 502 of needle 115 (806). The medical professional may align one or more electrodes 117 with one or more openings 507 by advancing, retracting, and/or rotating lead 106 relative to needle 115. One or more electrodes 117 and one or more openings 507 may be properly aligned when at least some of the one or more electrodes 117 are exposed to tissue of patient 102 through at least some of the one or more openings 507. In some examples, when properly aligned, at least some of electrodes 117 and at least some of openings 507 may have a same or substantially similar orientation and axial position relative to a longitudinal axis (e.g., longitudinal axis 504) of needle assembly 500.

The medical professional may control a medical device (e.g., IMD 104) to deliver one or more test stimulation signals via one or more electrodes 117 of lead 106 to tongue 118 of patient 102 (808). The medical professional may deliver test stimulation signals to tissue of patient 102 to stimulate hypoglossal nerve(s) and/or motor points (e.g., motor points 208A, 208B, 210A, and/or 210B) of patient 102 and detect response signals from tissue of patient 102. The one or more electrodes 117 of lead 106 may deliver the test stimulation signals to tissue of patient 102 through one or more openings 507 of needle 115.

The medical professional may detect electrical signals in tissue of patient 102 using needle assembly 500 (810). In some examples, the medical professional may control the medical device to receive electrical signals detected by needle assembly 500 and output information indicative of the received electrical signals. The medical professional may then determine a target treatment site based on the output information. Needle assembly 500 may detect electrical signals via one or more sensing electrodes (e.g., sensing electrodes 512, electrodes 117, or the like) positioned on lead 106 and/or needle 115. In some examples, the detected electrical signals may reflect the response of tissue of patient 102 (e.g., muscle contractions) in response to the test stimulation signals.

Based on the output information, the medical professional may determine if the medical device is detecting the expected responses to the test stimulation signals (812). The medical professional may determine that the expected responses are not detected if the outputted information is outside the boundaries of expected outputs for a given test stimulation signal. For examples, the outputted information may indicate that the amplitude, frequency, or other parameters of the detected electrical signal is greater than or less than the range of expected outputs.

If the medical professional determines that the medical device is not detecting expected responses (“NO” branch of 812), the medical professional may reposition needle assembly 500 within patient 102 (814). The medical professional may reposition needle assembly 500 further away from or closer to hypoglossal nerve(s) and/or motor points based on the differences between the expected responses and the actual outputted information. In some examples, the medical professional may determine, based on the outputted information, a target treatment site different from the target area and reposition needle assembly 500 within the target treatment site. Once needle assembly 500 is repositioned, the medical professional may delivery test stimulation signals via one or more electrodes 117 of lead 106 to tongue 118 of patient 102 (808) at the new position. The medical professional may repeat the process and iteratively reposition needle assembly 500 until the medical device detects the expected responses. If the medical professional determines that the medical device detects the expected responses (“YES” branch of 812), the medical professional may complete the implantation process (816).

The techniques of this disclosure may be implemented in a wide variety of computing devices, medical devices, or any combination thereof. Any of the described units, modules or components may be implemented together or separately as discrete but interoperable logic devices. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components.

The disclosure contemplates computer-readable storage media comprising instructions to cause a processor to perform any of the functions and techniques described herein. The computer-readable storage media may take the example form of any volatile, non-volatile, magnetic, optical, or electrical media, such as a RAM, ROM, NVRAM, EEPROM, or flash memory that is tangible. The computer-readable storage media may be referred to as non-transitory. A server, client computing device, or any other computing device may also contain a more portable removable memory type to enable easy data transfer or offline data analysis.

The techniques described in this disclosure, including those attributed to various modules and various constituent components, may be implemented, at least in part, in hardware, software, firmware or any combination thereof. For example, various aspects of the techniques may be implemented within one or more processors, including one or more microprocessors, DSPs, ASICs, FPGAs, or any other equivalent integrated, discrete logic circuitry, or other processing circuitry, as well as any combinations of such components, remote servers, remote client devices, or other devices. The term “processor” or “processing circuitry” may generally refer to any of the foregoing logic circuitry, alone or in combination with other logic circuitry, or any other equivalent circuitry.

Such hardware, software, firmware may be implemented within the same device or within separate devices to support the various operations and functions described in this disclosure. In addition, any of the described units, modules or components may be implemented together or separately as discrete but interoperable logic devices. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components. For example, any module described herein may include electrical circuitry configured to perform the features attributed to that particular module, such as fixed function processing circuitry, programmable processing circuitry, or combinations thereof.

The techniques described in this disclosure may also be embodied or encoded in an article of manufacture including a computer-readable storage medium encoded with instructions. Instructions embedded or encoded in an article of manufacture including a computer-readable storage medium encoded, may cause one or more programmable processors, or other processors, to implement one or more of the techniques described herein, such as when instructions included or encoded in the computer-readable storage medium are executed by the one or more processors. Example computer-readable storage media may include random access memory (RAM), read only memory (ROM), programmable read only memory (PROM), erasable programmable read only memory (EPROM), electronically erasable programmable read only memory (EEPROM), flash memory, a hard disk, a compact disc ROM (CD-ROM), a floppy disk, a cassette, magnetic media, optical media, or any other computer readable storage devices or tangible computer readable media. The computer-readable storage medium may also be referred to as storage devices.

In some examples, a computer-readable storage medium comprises non-transitory medium. The term “non-transitory” may indicate that the storage medium is not embodied in a carrier wave or a propagated signal. In certain examples, a non-transitory storage medium may store data that can, over time, change (e.g., in RAM or cache).

It should be noted that system 100, and the techniques described herein, may not be limited to treatment or monitoring of a human patient. In alternative examples, system 100 may be implemented in non-human patients, e.g., primates, canines, equines, pigs, and felines. These other animals may undergo clinical or research therapies that my benefit from the subject matter of this disclosure. Various examples are described herein, such as the following examples.

Example 1: a system comprising: a needle configured to percutaneously insert into skin and form a path for inserting a lead, the needle comprising: a pointed distal end for percutaneously inserting the needle for placement near a hypoglossal nerve of a patient; and an elongated body comprising an inner lumen, wherein the elongated body defines one or more openings connecting an outer surface of the elongated body to the inner lumen; and the lead configured to be disposed within the inner lumen of the elongated body, the lead comprising: a shaft; and one or more electrodes disposed on the shaft, configured to be placed near the hypoglossal nerve, and configured to stimulate the hypoglossal nerve for treating obstructive sleep apnea (OSA), wherein locations of the one or more electrodes on the shaft at least partially correspond to the one or more openings of the needle.

Example 2: the system of example 1, wherein the needle is configured for placement near the hypoglossal nerve and proximate to protrusor muscles in a tongue of the patient, and wherein the one or more electrodes are configured to be placed near the hypoglossal nerve and proximate to the protrusor muscles.

Example 3: the system of any of examples 1 and 2, wherein the needle is configured to percutaneously insert into the skin and form the path for inserting the lead without a guidewire or an introducer.

Example 4: the system of any of examples 1-3, wherein the elongated body further comprises an electrically conductive distal portion and an electrically insulated proximal portion.

Example 5: the system of example 4, wherein the electrically insulated proximal portion comprises an electrically insulated material and the electrically conductive distal portion comprises an electrically conductive material.

Example 6: the system of example 4, wherein the elongated body comprises a electrically conductive material, and wherein the electrically insulated proximal portion comprises an electrically insulated material disposed on the outer surface and disposed on the inner lumen of a proximal portion of the elongated body.

Example 7: the system of any of examples 5 and 6, wherein the electrically conductive material comprises a metallic alloy.

Example 8: the system of example 7, wherein the metallic alloy comprises stainless steel.

Example 9: the system of any of examples 5-8, wherein the electrically insulated material comprises a biocompatible polymer.

Example 10: the system of example 9, wherein the biocompatible polymer comprises parylene.

Example 11: the system of any of examples 1-10, wherein the lead further comprises a first reference mark configured to align the one or more electrodes with the one or more openings radially but not longitudinally, and a second reference mark configured to align the one or more electrodes with the one or more openings radially and longitudinally.

Example 12: the system of example 11, wherein the lead is configured to advance from the first reference mark to the second reference mark at a target area within a patient, and wherein the lead is further configured to deliver one or more electrical signals from at least one of the one or more electrodes to at least a portion of the target area through at least one of the one or more openings.

Example 13: the system of any of examples 1-12, wherein the one or more openings are positioned around a circumference of the elongated body at a same longitudinal position relative to a longitudinal axis of the elongated body.

Example 14: the system of any of examples 1-13, wherein the one or more openings comprises two or more openings, and wherein the two or more openings are disposed around a circumference of the inner lumen such that the two or more openings are equidistant from each other.

Example 15: the system of any of examples 1-14, wherein each of the one or more openings extends less than 180 degrees around a circumference of the elongated body.

Example 16: the system of any of examples 1-15, wherein each of the one or more openings at least partially corresponds to locations of at least one of the one or more electrodes.

Example 17: the system of any of examples 1-16, wherein the one or more electrodes comprises a ring electrode.

Example 18: the system of any of examples 1-17, wherein a distal end of the needle comprises a radiopaque element.

Example 19: the system of any of examples 1-18, wherein the one or more electrodes comprises one or more stimulation electrodes and one or more sensing electrodes, wherein the one or more stimulation electrodes are configured to deliver a stimulation signal to the hypoglossal nerve, and wherein the one or more sensing electrodes are configured to detect an evoked response following the delivery of the stimulation signal.

Example 20: a needle configured to percutaneously insert into skin and form a path for inserting a lead, the needle comprising: a pointed distal end for percutaneously inserting the needle for placement near a hypoglossal nerve of a patient; an elongated body comprising an inner lumen, the elongated body defining one or more openings defined by the elongated body, wherein the one or more openings are configured to connect an outer surface of the elongated body to the inner lumen, wherein the elongated body is configured to place the lead disposed within the inner lumen near the hypoglossal nerve, the lead configured to stimulate the hypoglossal nerve for treating obstructive sleep apnea (OSA), and wherein the one or more openings are configured to at least partially correspond to locations of one or more electrodes positioned on a shaft of the lead.

Example 21: the needle of example 20, wherein the needle is configured for placement near the hypoglossal nerve and proximate to protrusor muscles in a tongue of the patient, and wherein the one or more electrodes of the lead are configured to be placed near the hypoglossal nerve and proximate to the protrusor muscles.

Example 22: the needle of any of examples 20 and 21, wherein the needle is configured to percutaneously insert into the skin and form the path for inserting the lead without a guidewire or an introducer.

Example 23: the needle of any of examples 20-22, wherein the elongated body further comprises an electrically conductive distal portion and an electrically insulated proximal portion.

Example 24: the needle of example 23, wherein the electrically insulated proximal portion comprises an electrically insulated material and the electrically conductive distal portion comprises an electrically conductive material.

Example 25: the needle of example 23, wherein the elongated body comprises a electrically conductive material, and wherein the electrically insulated proximal portion comprises an electrically insulating material disposed on the outer surface and disposed on the inner lumen of a proximal portion of the elongated body.

Example 26: the needle of any of examples 24 and 25, wherein the electrically conductive material comprises a metallic alloy.

Example 27: the needle of example 26, wherein the metallic alloy comprises stainless steel.

Example 28: the needle of any of examples 24-27, wherein the electrically insulated material comprises a biocompatible polymer.

Example 29: the needle of example 28, wherein the biocompatible polymer comprises parylene.

Example 30: the needle of any of examples 20-29, wherein the elongated body is configured to retain the lead at a first position where the one or more openings are not aligned with the location of the one or more electrodes on the lead, and wherein the elongated body is further configured to retain the lead at a second position where the one or more openings of the elongated body are at least partially aligned with the location of the one or more electrodes on the lead.

Example 31: the needle of any of examples 20-30, wherein the one or more openings are positioned around a circumference of the elongated body at a same longitudinal position relative to a longitudinal axis of the elongated body.

Example 32: the needle of any of examples 20-31, wherein the one or more openings comprises two or more openings, and wherein the two or more openings are disposed around a circumference of the elongated body such that the two or more openings are equidistant from each other.

Example 33: the needle of any of examples 20-32, wherein each of the one or more openings extends less than 180 degrees around a circumference of the elongated body.

Example 34: the needle of any of examples 20-33, wherein a distal end of the needle comprises a radiopaque element.

Example 35: a method comprising: percutaneously inserting a needle into skin of a patient, the needle comprising an elongated body comprising an inner lumen and defining one or more openings connecting an outer surface of the elongated body to the inner lumen; navigating the needle proximate to a hypoglossal nerve of the patient; inserting a lead into the inner lumen of the elongated body, the lead configured to stimulate the hypoglossal nerve for treating obstructive sleep apnea (OSA), the lead comprising: a shaft; and one or more electrodes disposed on the shaft, wherein locations of the one or more electrodes at least partially correspond to the one or more openings of the needle; and placing the one or more electrodes near the hypoglossal nerve.

Example 36: The method of example 35, wherein placing the one or more electrodes near the hypoglossal nerve comprises: advancing the lead to a first position within the inner lumen of the needle, wherein the one or more electrodes of lead are obscured from the hypoglossal nerve by the needle at the first position: and advancing the lead to a second position within the inner lumen, wherein the one or more electrodes are at least partially exposed to the hypoglossal nerve of the patient through at least one of the one or more openings of the needle.

Example 37: the method of any of examples 35 and 36, wherein placing the one or more electrodes near the hypoglossal nerve comprises placing the needle near the hypoglossal nerve and proximate to protrusor muscles in a tongue of the patient.

Example 38: the method of any of examples 35-37, wherein percutaneously inserting the needle into the skin of the patient comprises percutaneously inserting the needle into the skin and forming a path for inserting the lead without a guidewire or an introducer.

Example 39: the method of any of examples 35-38, wherein the elongated body further comprises an electrically conductive distal portion and an electrically insulated proximal portion.

Example 40: the method of example 39, wherein the electrically insulated proximal portion comprises an electrically insulated material and the electrically conductive distal portion comprises an electrically conductive material.

Example 41: the method of example 39, wherein the elongated body comprises an electrically conductive material, and wherein the electrically insulated proximal portion comprises an electrically insulated material disposed on the outer surface and disposed on the inner lumen of a proximal portion of the elongated body.

Example 42: the method of any of examples 40 and 41, wherein the electrically conductive material comprises a metallic alloy.

Example 43: the method of example 42, wherein the metallic alloy comprises stainless steel.

Example 44: the method of any of examples 41-43, wherein the electrically insulated material comprises a biocompatible polymer.

Example 45: the method of example 44, wherein the biocompatible polymer comprises parylene.

Example 46: the method of any of examples 35-45, wherein: advancing the lead to the first position within the inner lumen of the needle comprises aligning a first reference mark on the lead with a reference point on the needle; and advancing the lead to the second position within the inner lumen comprises aligning a second reference mark on the lead with the reference point on the needle.

Example 47: the method of any of examples 35-46, wherein the one or more openings are positioned around a circumference of the elongated body at a same longitudinal position relative to a longitudinal axis of the elongated body.

Example 48: the method of any of examples 35-47, wherein the one or more openings comprises two or more openings, and wherein the two or more openings are disposed around a circumference of the inner lumen such that the two or more openings are equidistant.

Example 49: the method of any of examples 35-48, wherein each of the one or more openings extend less than 180 degrees around a circumference of the elongated body.

Example 50: the method of any of examples 35-49, wherein navigating the needle to the hypoglossal nerve further comprises determining a position of the needle within the patient using a radiopaque element positioned at a distal end of the needle.

Example 51: the method of any of examples 35-50, wherein the one or more electrodes comprises a ring electrode.

Example 52: the method of any of examples 35-51, further comprising: causing delivery, using one or more stimulation electrodes of the one or more electrodes, a stimulation signal to the hypoglossal nerve for evoking an evoked response.

Various examples have been described herein. Any combination of the described operations or functions is contemplated. These and other examples are within the scope of the following claims.

1. A system comprising:
a needle configured to percutaneously insert into skin and form a path for inserting a lead, the needle comprising:
a pointed distal end for percutaneously inserting the needle for placement near a hypoglossal nerve of a patient; and
an elongated body comprising an inner lumen, wherein the elongated body defines one or more openings connecting an outer surface of the elongated body to the inner lumen; and
the lead configured to be disposed within the inner lumen of the elongated body, the lead comprising:
a shaft; and
one or more electrodes disposed on the shaft, configured to be placed near the hypoglossal nerve, and configured to stimulate the hypoglossal nerve for treating obstructive sleep apnea (OSA), wherein locations of the one or more electrodes on the shaft at least partially correspond to the one or more openings of the needle.

2. The system of claim 1, wherein the needle is configured for placement near the hypoglossal nerve and proximate to protrusor muscles in a tongue of the patient, and wherein the one or more electrodes are configured to be placed near the hypoglossal nerve and proximate to the protrusor muscles.

3. The system of claim 1, wherein the elongated body further comprises an electrically conductive distal portion and an electrically insulated proximal portion.

4. The system of claim 1, wherein the lead further comprises a first reference mark configured to align the one or more electrodes with the one or more openings radially but not longitudinally, and a second reference mark configured to align the one or more electrodes with the one or more openings radially and longitudinally.

5. The system of claim 4, wherein the lead is configured to advance from the first reference mark to the second reference mark at a target area within a patient, and wherein the lead is further configured to deliver one or more electrical signals from at least one of the one or more electrodes to at least a portion of the target area through at least one of the one or more openings.

6. The system of claim 5, wherein the one or more openings are positioned around a circumference of the elongated body at a same longitudinal position relative to a longitudinal axis of the elongated body.

7. The system of claim 6, wherein each of the one or more openings at least partially corresponds to locations of at least one of the one or more electrodes.

8. The system of claim 7, wherein the one or more electrodes comprises one or more stimulation electrodes and one or more sensing electrodes, wherein the one or more stimulation electrodes are configured to deliver a stimulation signal to the hypoglossal nerve, and wherein the one or more sensing electrodes are configured to detect an evoked response following the delivery of the stimulation signal.

9. The system of claim 8, further comprising one or more fixation members disposed on the shaft of the lead, the one or more fixation members configured to expand radially outwards of the lead and penetrate tissue of the patient.

10. The system of claim 9, further comprising a sheath disposed within the inner lumen and around the lead, wherein the sheath comprises:
one or more openings configured to at least partially correspond to the one or more electrode disposed on the shaft of the lead; and
an electrically insulative material,
wherein the sheath is configured to retain the one or more fixation members in a collapsed configuration against the shaft of the lead, and wherein the sheath is configured to at least partially electrically insulate the lead from the needle.

11. The system of claim 1, further comprising:
a trocar configured to be disposed within the inner lumen of the elongated body of the needle, the trocar comprising:
one or more electrodes disposed on an elongated body of the trocar; and
a sheath disposed around the trocar, the sheath comprising:
an electrically insulative material; and
one or more openings configured to at least partially correspond to the one or more electrodes disposed on the elongated body of the trocar,
wherein the sheath is configured to at least partially electrically insulate the trocar from tissue of the patient.

12. A needle configured to percutaneously insert into skin and form a path for inserting a lead, the needle comprising:
a pointed distal end for percutaneously inserting the needle for placement near a hypoglossal nerve of a patient;
an elongated body comprising an inner lumen, the elongated body defining one or more openings defined by the elongated body, wherein the one or more openings are configured to connect an outer surface of the elongated body to the inner lumen, wherein the elongated body is configured to place the lead disposed within the inner lumen near the hypoglossal nerve, the lead configured to stimulate the hypoglossal nerve for treating obstructive sleep apnea (OSA), and wherein the one or more openings are configured to at least partially correspond to locations of one or more electrodes positioned on a shaft of the lead.

13. The needle of claim 12, wherein the needle is configured for placement near the hypoglossal nerve and proximate to protrusor muscles in a tongue of the patient, and wherein the one or more electrodes of the lead are configured to be placed near the hypoglossal nerve and proximate to the protrusor muscles.

14. The needle of claim 13, wherein the elongated body further comprises an electrically conductive distal portion and an electrically insulated proximal portion.

15. The needle of claim 14, wherein the elongated body is configured to retain the lead at a first position where the one or more openings are not aligned with the location of the one or more electrodes on the lead, and wherein the elongated body is further configured to retain the lead at a second position where the one or more openings of the elongated body are at least partially aligned with the location of the one or more electrodes on the lead.

16. A method comprising:
percutaneously inserting a needle into skin of a patient, the needle comprising an elongated body comprising an inner lumen and defining one or more openings connecting an outer surface of the elongated body to the inner lumen;
navigating the needle proximate to a hypoglossal nerve of the patient;
inserting a lead into the inner lumen of the elongated body, the lead configured to stimulate the hypoglossal nerve for treating obstructive sleep apnea (OSA), the lead comprising:
a shaft; and
one or more electrodes disposed on the shaft, wherein locations of the one or more electrodes at least partially correspond to the one or more openings of the needle; and
placing the one or more electrodes near the hypoglossal nerve.

17. The method of claim 16, wherein placing the one or more electrodes near the hypoglossal nerve comprises:
advancing the lead to a first position within the inner lumen of the needle, wherein the one or more electrodes of lead are obscured from the hypoglossal nerve by the needle at the first position; and
advancing the lead to a second position within the inner lumen, wherein the one or more electrodes are at least partially exposed to the hypoglossal nerve of the patient through at least one of the one or more openings of the needle.

18. The method of claim 17, wherein placing the one or more electrodes near the hypoglossal nerve comprises:
expanding one or more fixation members disposed on the shaft of the lead radially outwards of the lead; and
penetrating tissue of the patient with the one or more fixation members.

19. The method of claim 18, wherein expanding the one or more fixation members disposed on the shaft of the lead radially outwards of the lead comprises:
retracting a sheath proximal to the shaft of the lead; and
releasing the one or more fixation members retained by the sheath, wherein the sheath comprises:
an electrically insulative material; and
one or more openings at least partially corresponding to the one or more electrodes disposed on the shaft of the lead,
wherein the sheath at least partially electrically insulates the lead from the needle.

20. The method of claim 19, further comprising:
advancing a trocar disposed within the inner lumen of the needle from a distal tip of the needle, wherein the trocar comprises:
an elongated body of the trocar; and
one or more electrodes disposed on the elongated body of the trocar,
wherein the trocar is disposed within a sheath, the sheath comprising:
an electrically insulative material; and
one or more openings at least partially corresponding to the one or more electrodes disposed on the elongated body of the trocar; and
delivering an electrical signal to the hypoglossal nerve via the trocar.
